(19) United States
(12) Patent Application Publication
(10) Pub. No.: US 2023/0056661 A1
(43) Pub. Date: Feb. 23, 2023

(54) METHODS AND COMPOSITIONS FOR PRODUCTION OF XENOGENEIC ISLET CELLS AND TREATMENT OF INSULIN-RESISTANT OR -DEFICIENT CONDITIONS WITH THE SAME

(71) Applicants: eGenesis, Inc., Cambridge, MA (US); Hangzhou Qihan Biotechnology Co., Ltd., Hangzhou, Zhejiang (CN)

(72) Inventors: Yangbin GAO, Hangzhou (CN); Yanan YUE, Hangzhou (CN); Luhan YANG, Cambridge, MA (US); Marc GUELL, Barcelona (ES); Yinan KAN, Cambridge, MA (US); Wenning QIN, Cambridge, MA (US)

(21) Appl. No.: 17/790,797

(22) PCT Filed: Jan. 7, 2021
(86) PCT No.: PCT/CN2021/070659

§ 371 Date: Jul. 5, 2022

(30) Foreign Application Priority Data
Jan. 7, 2020 (CN) PCT/CN2020/070698

(51) Int. Cl. C12N 5/071; A61P 3/10; A61K 35/39; A61K 39/395; A61K 31/436; A61K 31/522; A61K 31/4402; A61K 31/573; A61K 38/28

(52) U.S. Cl. CPC C12N 5/0676; A61P 3/10; A61K 35/39; C12N 5/0677; A61K 39/3955; A61K 31/436; A61K 31/522; A61K 31/4402; A61K 31/573; A61K 38/28; C12N 2510/00; C12N 2509/00; C12N 2509/10; C12N 2501/999

(57) ABSTRACT

Described here are methods, compositions, and systems for generating transgenic islet cells suitable for xenotransplantation.

### CROSS-REFERENCE TO RELATED APPLICATIONS

This application claims priority to International Application No. PCT/CN2020/070698, filed Jan. 7, 2020, which is incorporated by reference herein in its entirety for all purposes.

### BACKGROUND OF THE INVENTION

Current estimates indicate the prevalence of type 1/2 diabetes will reach 4.4% for all age groups worldwide by 2030. Currently utilized pharmacological treatments for type 1 diabetes include insulin replacement, and for type 2 diabetes include insulin supplementation, either alone or in combination with metformin, sulfonylureas, glinides, DPP-4 inhibitors, GLP-1 receptor agonists, SGLT-2 inhibitors, or pioglitazone. All of these strategies require detailed patient management and medication compliance. Additionally, many patients fail to achieve glycemic control despite these interventions.

There is a need for therapeutic strategies that improve glucose control in diabetics in the absence of complicated administration of antidiabetic drugs or insulin, or that improve glucose control in diabetics that have poor glucose control despite administration of antidiabetic drugs or insulin. Allogeneic islet cell transplantation (e.g. intraportal islet cell transplantation) has seen increased usage in the case of type 1 diabetes patients with severe risk factors (e.g. unstable T1DM, hypoglycemia unawareness, severe hypoglycemic episodes, glycemic lability); however, the necessity of stringent immunosuppression, graft survival challenges, and donor cell availability hamper wider usage of this technique in type 1 diabetes patients, type 2 diabetes patients, and type 1 or 2 diabetes patients early in the disease when improved glucose control most minimizes the risk of long-term complications.

### SUMMARY OF THE INVENTION

In an aspect, the present disclosure provides an isolated transgenic porcine islet cell, wherein the cell: (a) is substantially free of enzymatic activity of at least one glycosyltransferase enzyme, wherein the glycosyltransferase enzyme is GGTA, B4GALNT2, or CMAH; (b) expresses at least two polypeptide sequences derived from a non-porcine mammalian species, wherein the at least two polypeptide sequences comprise at least two of CD46, CD55, CD59, THBD, TFPI, CD39, B2M, HLAE, CD47, A20, PD-L1, FASL, or HO-2; and (c) exhibits one or more of the following: reduced toxicity from complement derived from the non-porcine mammalian species, reduced induction of activated protein C coagulation derived from the non-porcine mammalian species, reduced induction of thrombin-antithrombin complex derived from the non-porcine mammalian species, or reduced toxicity from NK cells derived from the non-porcine species.

In some embodiments, the cell is substantially free of enzymatic activity of at least two, or all three glycosyltransferase enzymes selected from GGTA, B4GALNT2, and CMAH.

In some embodiments of any of the isolated transgenic porcine islet cell disclosed herein, the cell expresses at least three, at least four, at least five, at least six, at least seven, at least eight, at least nine, at least ten, at least eleven, at least twelve, or all of CD46, CD55, CD59, THBD, TFPI, CD39, B2M, HLAE, CD47, A20, PD-L1, FASL, or HO-1.

In another aspect, the present disclosure provides an isolated transgenic porcine islet cell, wherein the islet cell: (a) is substantially free of enzymatic activity of at least two glycosyltransferase enzymes, wherein the glycosyltransferase enzymes comprise at least two of GGTA, B4GALNT2, or CMAH; (b) expresses a polypeptide sequence derived from a non-porcine mammalian species, wherein the polypeptide sequence is CD46, CD55, CD59, THBD, TFPI, CD39, B2M, HLAE, CD47, A20, PD-L1, FASL, or HO-1; and (c) exhibits reduced toxicity from complement derived from the non-porcine mammalian species, reduced induction of activated protein C coagulation derived from the non-porcine species, reduced induction of thrombin-antithrombin complex derived from the non-porcine species, or reduced toxicity from NK T-cells derived from the non-porcine species.

In some embodiments, the cell is substantially free of enzymatic activity of GGTA, B4GALNT2, and CMAH.

In some embodiments of any of the isolated transgenic porcine islet cell disclosed herein, the cell expresses at least two, at least three, at least four, at least five, at least six, at least seven, at least eight, at least nine, at least ten, at least eleven, at least twelve, or all of CD46, CD55, CD59, THBD, TFPI, CD39, B2M, HLAE, CD47, A20, PD-L1, FASL, or HO-1.

In some embodiments of any of the isolated transgenic porcine islet cell disclosed herein, the cell expresses CD46, CD55, CD59, CD39, B2M, HLAE, and CD47.

In some embodiments of any of the isolated transgenic porcine islet cell disclosed herein, the cell is substantially free of expression of the glycosyltransferase enzyme or enzymes. In some embodiments, the cell comprises a frameshift mutation in the glycosyltransferase enzyme or enzymes resulting in premature termination of translation, thereby ablating activity of the glycosyltransferase enzyme.

In some embodiments of any of the isolated transgenic porcine islet cell disclosed herein, a nucleic acid sequence or sequences encoding the polypeptide sequence or sequences derived from non-porcine mammalian species are inserted within non-orthologous loci of the porcine ortholog.

In some embodiments of any of the isolated transgenic porcine islet cell disclosed herein, a nucleic acid sequence or sequences encoding the polypeptide sequence or sequences derived from non-porcine mammalian species are operably linked to non-orthologous promoters of the porcine ortholog. In some embodiments, the non-orthologous promoters are non-porcine promoters.

In some embodiments of any of the isolated transgenic porcine islet cell disclosed herein, the islet cell is derived by disaggregation of a porcine pancreas.

In some embodiments of any of the isolated transgenic porcine islet cell disclosed herein, the islet cell is an alpha cell, a beta cell, a delta cell, an epsilon cell, a Pancreatic polypeptide (PP) cell, or any combination thereof.

In some embodiments of any of the isolated transgenic porcine islet cell disclosed herein, the non-porcine mammalian species is a primate species.

In some embodiments of any of the isolated transgenic porcine islet cell disclosed herein, the cell exhibits survival greater than 8 days when transplanted into the non-porcine mammalian species.

In some embodiments of any of the isolated transgenic porcine islet cell disclosed herein, the cell exhibits a reduced IBMIR to PBMCs isolated from the non-porcine mammalian species.

In another aspect, the present disclosure provides a composition comprising a therapeutically effective amount of any of the isolated transgenic porcine islet cell disclosed herein. In some embodiments, the isotonic buffered solution further comprises heparin or a TNF-alpha inhibitor.

In some embodiments of any of the composition disclosed herein, the composition comprises at least about 12% to about 25% beta cells or at least about 15% to about 30% alpha cells.

In some embodiments of any of the composition disclosed herein, the composition is prepared according to any one of the methods disclosed herein.

In another aspect, the present disclosure provides a method of treating an insulin resistant or deficient condition in a non-porcine mammal in need thereof, comprising administering a therapeutically effective dose of any of the isolated transgenic porcine islet cell disclosed herein or any of the composition disclosed herein to the mammal.

In some embodiments, the method comprises centrally administering the cells via an internal jugular vein or a hepatic portal vein of the mammal.

In some embodiments of any one of the methods disclosed herein, the insulin resistant condition comprises type 1 diabetes mellitus.

In some embodiments of any one of the methods disclosed herein, the insulin resistant condition comprises type 2 diabetes mellitus.

In some embodiments of any one of the methods disclosed herein, the non-porcine mammal has received an induction regimen comprising therapeutically effective doses of anti-thymocyte globulin, anti-CD40 antibody, anti-CD20 antibody, a rapalog, a calcineurin inhibitor, ganciclovir or a prodrug thereof, an antihistamine, and a corticosteroid prior to administering the transgenic porcine islet cell or the composition.

In some embodiments of any one of the methods disclosed herein, the method further comprises administering therapeutically effective doses of anti-CD40 antibody, a rapalog, a calcineurin inhibitor, and ganciclovir or a prodrug thereof following administration of the transgenic porcine islet cell or the composition.

In some embodiments of any one of the methods disclosed herein, the method further comprises administering therapeutically effective doses of an intermediate- or long-acting insulin analog, insulin glargine, insulin detemir, or NPH insulin following administration of the transgenic porcine islet cell or the composition.

In some embodiments of any one of the methods disclosed herein, any of the therapeutically effective doses disclosed herein is at least 5,000 IEQ per kg of non-porcine mammal body weight.

In another aspect, the present disclosure provides an isolated porcine islet comprising any of the isolated transgenic porcine islet cell disclosed herein. In some embodiments, the islet is substantially free of pancreatic exocrine cells

In another aspect, the present disclosure provides an isolated porcine pancreatic organoid comprising any of the isolated transgenic porcine islet cell disclosed herein. In some embodiments, the organoid is substantially free of pancreatic exocrine cells. In some embodiments, the pancreatic organoid is prepared by: (a) isolating a pancreas from a neonatal porcine animal on neonatal day 7 or earlier; and (b) subjecting the pancreas to mechanical or enzymatic digestion to generate organoid fragments, and optionally: (c) purifying organoid fragments of step (b) by ficoll gradient sedimentation.

In another aspect, the present disclosure provides an isolated porcine pancreas comprising any of the isolated transgenic porcine islet cell disclosed herein.

In another aspect, the present disclosure provides a method of improving yield of islets from a porcine donor prior to transplantation to a non-porcine mammalian recipient, comprising: (a) providing pancreatic organoids from a neonatal porcine animal that have been subjected to a purification procedure; (b) culturing the organoids in the presence of an effective concentration of a caspase inhibitor for at least 90 minutes following the purification; and (c) continuing culture in the presence of an effective concentration of a corticosteroid for at least 7 days.

In some embodiments, the purification procedure comprises: (a) isolating a pancreas from a transgenic neonatal porcine animal on neonatal day 7 or earlier; and (b) subjecting the pancreas to mechanical or enzymatic digestion to generate organoid fragments, and optionally: (c) purifying organoid fragments from the digested pancreas by ficoll gradient sedimentation.

In some embodiments of any one of the methods disclosed herein, the neonatal porcine animal is a transgenic pig comprising at least one porcine cell according to any of the isolated transgenic porcine islet cell disclosed herein.

In some embodiments of any one of the methods disclosed herein, the caspase inhibitor is Z-VAD-FMK.

In some embodiments of any one of the methods disclosed herein, the corticosteroid is methylprednisolone.

In some embodiments of any one of the methods disclosed herein, the pancreatic organoids are cultured in the presence of IBMX, a phosphodiesterase inhibitor, or an adenosine receptor antagonist.

In some embodiments of any one of the methods disclosed herein, the pancreatic organoids are cultured in the presence of nicotinamide or a metabolically acceptable analog thereof.

In another aspect, the present disclosure provides a method of treating an insulin resistant or deficient condition in a non-porcine mammal in need thereof, comprising transplanting organoids according to any one of the organoids disclosed herein into the non-porcine mammal when the organoids meet any of the following criteria: (a) endotoxin less than about 5 EU/kg; (b) negative gram stain; (c) viability greater than about 70%; or (d) islet concentration greater than or equal to about 20,000 IEQ/mL of total settled volume.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 (FIG. 1) depicts FACS immunostaining results of 4-7 transgenic endothelial umbilical vein porcine cells (PUVECs) incubated in human serum. The top panel is a FACS plot showing staining by either IgG or IgM of human umbilical vein endothelial cells (“HUVEC”), transgenic 4-7 porcine umbilical vein endothelial cells (“4-7 PUVEC”), or normal porcine umbilical vein endothelial cells (“WT PUVEC”). Transgenic 4-7 PUVECs show diminished binding of IgG and IgM antibodies from human serum versus their normal pig counterparts, similar to HUVEC cells. Data are shown as mean±standard deviation. Error bars indicate standard deviation and P-values are derived from unpaired, two-tailed Student's t-test. * denote that P<0.05; ** denote that P<0.01.

FIG. 2 (FIG. 2) depicts results of human complement toxicity assays performed on 4-7 transgenic endothelial umbilical vein porcine cells (PUVECs). The left panel is a diagram illustrating the assay workflow, whereas the right panel is a chart illustrating the death of either human umbilical vein endothelial cells (“HUVEC”), transgenic 4-7 porcine umbilical vein endothelial cells (“4-7 PUVEC”), or normal porcine endothelial cells (“WT PUVEC”) after incubation with various concentrations of human complement (“HC”). 4-7 cells show dramatically decreased death in response to human complement versus their normal pig counterparts, similar to human HUVEC cells.

FIG. 3 (FIG. 3) depicts results of analyses performed to validate expression/functionality of CD39 in 4-7 transgenic porcine umbilical vein endothelial porcine cells (PUVECs). Transgenic 4-7 porcine umbilical vein endothelial cells (“4-7 PUVEC”) have significantly higher ADPase biochemical activity of hCD39 as measured by phosphate production when incubated with ADP compared to HUVECs and WT PUVECs. Data are shown as mean±standard deviation. Error bars indicate standard deviation and P-values are derived from unpaired, two-tailed Student's t-test. ** denote that P<0.01.

FIG. 4 (FIG. 4) shows a schematic for an activated protein C assay in xenogeneic cells using human protein C and human thrombin.

FIG. 5 (FIG. 5) shows results of a thrombin-antithrombin III (TAT) formation assay on 4-7 cells. The left panel is a diagram showing the workflow for measuring thrombin-antithrombin III (TAT) complex formation using human blood, whereas the right panel is a chart depicting results of the corresponding assay with HUVECs, 4-7 PUVECs, or WT PUVECs. 4-7 cells show reduced TAT formation compared to WT PUV ECs, comparable to HUVEC cells.

FIG. 6 (FIG. 6) depicts results of a platelet lysis assay performed on 4-7 transgenic cells. Shown are FACS traces quantitating the number of platelets remaining (outlined cluster) from human blood after incubation with HUVECs, 4-7 PUVECs, or WT PUVECs for 45 or 60 minutes. 4-7 cells continue to show elevated fractions of platelets remaining relative to porcine WT PUVECs, which is comparable to the fraction of platelets remaining when incubated with HUVEC cells.

FIG. 7 (FIG. 7) quantitates the results of the experiment shown in FIG. 6 at additional timepoints (5 minutes, 15 minutes) as remaining CD41-positive platelets (MFI indicates the mean fluorescence intensity in CD41 channel by FACs analysis).

FIG. 8 (FIG. 8) depicts results of NK cell toxicity assays performed on 4-7 transgenic cells. Shown are charts depicting the results of NK toxicity assays performed at an effector:target cell ratio of 10 on HUVECs, 4-7 PUVECs, or WT PUVECs. 4-7 PUVECS show intermediate cell killing values between normal PUVECs and HUVEC cells.

FIG. 9 (FIG. 9) is a chart depicting an example workflow for processing porcine islet cells for transplantation. In an example embodiment, neonatal pigs are subjected to pancreatectomy (“procurement”), after which the pancreas is chopped and digested in collagenase (“islet isolation”). The digested islet cells are then transferred to a gas-permeable, water-impermeable bag and held at 22-24 dC until they can be cultured (“transportation”). Islet cells are then cultured for a period of time (optionally with EGM2 medium and a caspase inhibitor, “culture”) before being subjected to quality control procedures such as functional islet equivalent quantitation (IEQ), endotoxin assays, gram staining, viability assays, and cell purity assays.

FIG. 10 (FIG. 10) depicts results of an islet isolation procedure according to FIG. 9 performed on transgenic (4-7) or normal (WT) Bana minipigs.

FIG. 11 (FIG. 11) depicts results of platelet lysis or TAT complex formation assays performed on islet cells isolated as in FIG. 9. Left panel shows that 4-7 islets reveal decreased platelet lysis compared to WT islets and experimental control (NC, saline only) when incubated with whole human blood. Right panel shows that the 4-7 islets reveal reduced formation of TAT complex compared to WT islets and experimental control (NC, saline only) when incubated with whole human blood.

FIG. 12 (FIG. 12) depicts results of instant blood-mediated inflammatory reaction (IBMIR) assays performed with human blood on 4-7 islets derived as in FIG. 9. Shown are IHC micrographs at 200× magnification showing staining for antibody (IgG and IgM, left panel) and complement (C3a and C4 d, right panel) foci after incubation of 4-7 islet sections with human blood. 4-7 islet cells show decreased staining and foci associated with IgG, IgM, C3a, and C4 d, indicating the islet cells show reduced IBMIR.

FIG. 13 (FIG. 13) depicts neutrophil infiltration into 4-7 islets, WT islets, and experimental control (NC, saline only) after incubation with human blood as in FIG. 12. 4-7 islets reveal higher numbers of remaining neutrophils compared to WT islets and experimental control.

FIG. 14 (FIG. 14) depicts islet cells isolated as in FIG. 9 over time under 2 different culture conditions. Shown is a graph depicting islet equivalents (IEQ) over 7 days culture in either EGM-2 medium or standard medium (“F-10”, denoting Ham's F-10) medium. EGM-2 medium was associated with an improved yield of islets.

FIG. 15 (FIG. 15) depicts islet cells isolated as in FIG. 9 over time under 2 different culture conditions: F-10 culture media and NEO culture media.

FIG. 16 (FIG. 16) compares culture of islets isolated as in FIG. 9 in medium without corticosteroid (left panel) versus medium with corticosteroid (right panel). Corticosteroid was associated with an improved yield of islets.

FIG. 17 (FIG. 17) compares cell fractions in islets isolated as in FIG. 9 either under initial (top row, in F-10 media) or improved (EGM-2 medium+corticosteroid, bottom row) culture conditions. Shown are FACS traces comparing intact islet cells (left), beta cells (middle), or living beta cells (right) between the two conditions. The improved condition was associated with improved numbers of intact islet cells and improved numbers of beta cells.

FIG. 18 (FIG. 18) shows protein expression validation of 4-7 transgenes in kidney cryosections by immunofluorescence staining. Scale bars (white), 75 μm.

FIG. 19 (FIG. 19) shows blood glucose of NCG mice receiving STZ followed by islet transplant with WT neonatal porcine islets over a period of 60 days, demonstrating that blood glucose normalizes after ˜40 days. Immunofluorescence staining validation of 3 knockouts and 9 transgenes in 4-7 kidney cryosections. Antibodies Scale bars (white), 75 μm.

FIG. 20 (FIG. 20) shows blood glucose of NCG mice receiving STZ followed by islet transplant with WT porcine islet cells (“WT Tx”), 4-7 islet cells (“4-7 Tx”), or a sham operation (“Sham Tx”) over a period of 126 days. Different islet doses, including 4,000 IEQ; 2,000 IEQ; and 1,000 IEQ, were applied for both “4-7 Tx” and “WT Tx” experimental groups.

FIG. 21 (FIG. 21) shows a typical induction, immunosuppression, transplant and management protocol for NHP transplanted with islets according to the methods described herein.

FIG. 22 (FIG. 22) shows an example response of a glucose tolerance test of a NHP in terms of blood glucose, insulin, and C-peptide pre- and post-STZ induction of diabetes according to the methods described herein, demonstrating that the protocol successfully induces diabetes in the animals.

FIG. 23 (FIG. 23), FIG. 24 (FIG. 24), and FIG. 25 (FIG. 25) show WBC and lymphocyte count (FIG. 23), CD4+ cell type/CD8+ cell type/B cell/NK cell counts (FIG. 24) and rapamycin levels (FIG. 25) of the animals described in Table 2 over up to 70 days post-islet transplant.

FIG. 26 (FIG. 26) shows hematoxylin/eosin stains and anti-chromogranin A staining of liver biopsies for animals MA-1 and MA-2 12 hr and Imo post-transplant demonstrating presence of islets in liver tissue.

FIG. 27 (FIG. 27) shows immunofluorescence staining analysis of liver biopsies for animal MB-11 24 hr post-transplant demonstrating presence of islets (as revealed by positive signal of insulin and glucagon staining) in liver tissue. Monkey IgG, CD41 (a marker of platelets), fibrinogen (a marker for the indication of coagulation) and CD68 (a marker of macrophage) were also detected around the insulin-positive WT porcine islets, indicating the occurrence of instant blood-mediated inflammatory reaction (IBMIR) at 24-h post-transplantation in MB-11. This result also indicates that genetic modification is essential to enhance the survival of porcine islets in vivo.

FIG. 28 (FIG. 28) shows the serum concentrations of porcine c-peptide, monkey c-peptide, fasting blood glucose, and exogenous insulin intake of the monkey recipient at different post-transplantation time points using WT pig islets. Porcine c-peptide can be steadily detected in animal serum within 55 days after porcine islet transplantation.

### DETAILED DESCRIPTION OF THE INVENTION

### Overview

The present disclosure addresses the immunosuppression, graft survival, and donor cell availability challenges associated with transplantation by providing xenogeneic islet cells for transplantation. The disclosed xenogeneic cells (e.g. genetically modified xenogeneic cells) exhibit decreased immunogenicity and increased survival and thus are suitable for islet cell transplantation, requiring reduced use of immunosuppressants in transplant recipients. Further described herein are methods, compositions, and systems for deriving such cells, as well as therapeutic methods involving the use of such cells.

### Definitions

The terms “pig”, “swine” and “porcine” are used herein interchangeably to refer to anything related to the various breeds of domestic pig, species Sus scrofa.

The terms “treatment,” “treating,” “alleviation” and the like, when used in the context of a disease, injury or disorder, are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The term “biologically active” when used to refer to a fragment or derivative of a protein or polypeptide means that the fragment or derivative retains at least one measurable and/or detectable biological activity of the reference full-length protein or polypeptide. For example, a biologically active fragment or derivative of a CRISPR/Cas9 protein may be capable of binding a gRNA, sometimes also referred to herein as a single guide RNA (sgRNA), binding a target DNA sequence when complexed with a guide RNA, and/or cleaving one or more DNA strands. For example, a biologically active fragment or derivative of a cell receptor may be capable of binding the natural ligand that signals through said receptor or be capable of transmitting an intracellular signal generally transmitted by said receptor in response to ligand.

As used herein, the term “indel” herein refers to an insertion or deletion of nucleotide bases in a target DNA sequence in a chromosome or episome. Such an insertion or deletion may be of 1, 2, 3, 4, 5, 6, 7, 8, 9, 10, or more bases, for example. An indel in certain embodiments can be even larger, at least about 20, 30, 40, 50, 60, 70p, 80, 90, or 100 bases. If an indel is introduced within an open reading frame (ORF) of a gene, the indel may disrupt wild type expression of protein encoded by the ORF by creating a frameshift mutation. An indel may be the result of double-stranded cleavage of a genomic sequence (e.g. by a site-directed or programmable nuclease), followed by cellular repair using non-homologous end-joining (NHEJ).

As used herein, the term “type 1 diabetes mellitus” (T1DM) refers to a condition characterized by an inability to produce insulin due to destruction (e.g. autoimmune destruction) of the beta cells in the pancreas. In some embodiments, type 1 diabetes mellitus is defined by particular clinical criteria (“stage 3 T1DM”), including at least one of a fasting plasma glucose (FPG) level ≥126 mg/dL (7.0 mmol/L), a 2-hour plasma glucose level ≥200 mg/dL (11.1 mmol/L) during a 75-g oral glucose tolerance test (OGTT), a random plasma glucose ≥200 mg/dL (11.1 mmol/L) in a patient with classic symptoms of hyperglycemia or hyperglycemic crisis, or a hemoglobin A1c (HbA1c) level of 6.5% or higher. In some embodiments, “type 1 diabetes mellitus” (T1DM) is a particular stage of T1DM, such as stage 1, stage 2, or stage 3. While stage 1 can be asymptomatic except for the presence of multiple autoantibodies against beta cells, stage 2 can be accompanied by dysglycemia (IFG and/or IGT), intermediate FPG levels such as 100-125 mg/dL (5.6-6.9 mmol/L), intermediate 2-hour plasma glucose levels 140-199 mg/dL (7.8-11.0 mmol/L) during a 75-g oral glucose tolerance test (OGTT), or an intermediate hemoglobin A1c (HbA1c) level of 5.7-6.4% (39-47 mmol/mol). Type 1 diabetes mellitus (T1DM) may occur in children, juveniles, adolescents, or adults. Type 1 diabetes is typically diagnosed following an incident of polyuria, polydipsia, polyphagia, diabetic ketoacidosis, or unexplained weight loss.

As used herein, the term “type 2 diabetes mellitus” (T2DM) refers to a condition characterized by progressive loss of p-cell insulin secretion frequently on the background of insulin resistance. In some embodiments, type 1 diabetes mellitus is defined by particular clinical criteria, including at least one of a fasting plasma glucose (FPG) level ≥126 mg/dL (7.0 mmol/L), a 2-hour plasma glucose level ≥200 mg/dL (11.1 mmol/L) during a 75-g oral glucose tolerance test (OGTT), a random plasma glucose ≥200 mg/dL (11.1 mmol/L) in a patient with classic symptoms of hyperglycemia or hyperglycemic crisis, or a hemoglobin A1c (HbA1c) level of 6.5% or higher.

In some embodiments, proteins or genes referred to herein are according to the following table:

| | | | Example |
|---|---|---|---|
| | | | Human or Pig |
| Protein/ | | | UniProtKB |
| Gene | Name | Also Known As | reference |
| GGTA | N-acetyl- | GGTA1 | P50127 |
| | lactosaminide | | (GGTA1_PIG) |
| | alpha-1,3-galacto- | | |
| | syltransferase | | |
| β4GALNT2 | β1,4 N-acetyl- | B4GALNT2, | |
| | galactosaminyl | B4GAL | |
| | transferase | | |
| CMAH | Cytidine | | O19074 |
| | monophosphate- | | (CMAH_PIG) |
| | N-acetylneura- | | |
| | minic acid | | |
| | hydroxylase | | |
| CD46 | CD46 | | P15529 |
| | complement | | (MCP_HUMAN) |
| | regulatory protein | | |
| CD55 | CD55 Molecule | Decay- | P08174 |
| | (Cromer Blood | Accelerating | (DAF_HUMAN) |
| | Group) | Factor, DAF | |
| CD59 | CD59 Molecule | | P13987 |
| | (CD59 Blood | | (CD59_HUMAN) |
| | Group) | | |
| THBD | Thrombomodulin | CD141 Antigen | P07204 |
| | | | (TRBM_HUMAN) |
| TFPI | Tissue factor | Lipoprotein- | P10646 |
| | pathway inhibitor | Associated | (TFPI1_HUMAN) |
| | | Coagulation | |
| | | Inhibitor | |
| CD39 | CD39 antigen | Ectonucleoside | P55772 |
| | | triphosphate | (ENTP1_MOUSE) |
| | | diphosphohydro- | |
| | | lase 1, ENTPD1 | |
| HLA-E | Major | MHC Class I | P13747 |
| | Histo- | Antige nE, MHC | (HLAE_HUMAN) |
| | compatibility | Class Ib Antigen | |
| | Complex, Class | | |
| | I, E | | |
| B2M | Beta-2- | Beta Chain Of | P61769 |
| | Microglobulin | MHC Class I | (B2MG_HUMAN) |
| | | Molecules | |
| CD47 | Cluster of | integrin | Q08722 |
| | Differentiation | associated | (CD47_HUMAN) |
| | 47, CD47 | protein | |
| | Antigen | | |
| A20 | A20 | TNF Alpha | P21580 |
| | | Induced Pro- | (TNAP3_HUMAN) |
| | | tein 3, TNFAIP3 | |
| PD-L1 | Programmed cell | CD274, B7 | Q9NZQ7 |
| | death 1 ligand 1 | Homolog 1, | (PD1L1_HUMAN) |
| | | B7H1 | |
| FasL | Fas Ligand | FASL, CD95, | P48023 |
| | | Tumor Necrosis | (TNFL6_HUMAN) |
| | | Factor (Ligand) | |
| | | Superfamily, | |
| | | Member 6, | |
| | | TNFL6 | |

### Cells, Tissues, Methods Generating Cells and Tissues, and Methods of Treatment Using Such

The present disclosure provides cells, tissues, and organs having multiple modified genes, and methods of generating the same. In some embodiments, the cells, tissue, or organs, are obtained from an animal. In some embodiments, the animal is a mammal. In some embodiments, the mammal is a non-human mammal, for example, equine, primate, porcine, bovine, ovine, caprine, canine, or feline. In some embodiments, the mammal is a porcine.

In some embodiments, the one or more cells is a porcine cell. Non-limiting examples of the breeds from which a porcine cell originates or is derived include any of the following pig breeds: American Landrace, American Yorkshire, Aksai Black Pied, Angeln saddleback, Appalachian English, Arapawa Island, Auckland Island, Australian Yorkshire, Babi Kampung, Ba Xuyen, Bantu, Basque, Bazna, Beijing Black, Belarus Black Pied, Belgian Landrace, Bengali Brown Shannaj, Bentheim Black Pied, Berkshire, Bisaro, Bangur, Black Slavonian, Black Canarian, Breitovo, British Landrace, British Lop, British Saddleback, Bulgarian White, Cambrough, Cantonese, Celtic, Chato Murciano, Chester White, Chiangmai Blackpig, Choctaw Hog, Creole, Czech Improved White, Danish Landrace, Danish Protest, Dermantsi Pied, Li Yan, Duroc, Dutch Landrace, East Landrace, East Balkan, Essex, Estonian Bacon, Fengjing, Finnish Landrace, Forest Mountain, French Landrace, Gascon, German Landrace, Gloucestershire Old Spots, Gottingen minipig, Grice, Guinea Hog, Hampshire, Hante, Hereford, Hezuo, Hogan Hog, Huntington Black Hog, Iberian, Italian Landrace, Japanese Landrace, Jeju Black, Jinhua, Kakhetian, Kele, Kemerovo, Korean Native, Krskopolje, Kunekune, Lamcombe, Large Black, Large Black-White, Large White, Latvian White, Leicoma, Lithuanian Native, Lithuanian White, Lincolnshire Curly-Coated, Livny, Malhado de Alcobaca, Mangalitsa, Meishan, Middle White, Minzhu, Minokawa Buta, Mong Cai, Mora Romagnola, Moura, Mukota, Mulefoot, Murom, Myrhorod, Nero dei Nebrodi, Neijiang, New Zealand, Ningxiang, North Caucasian, North Siberian, Norwegian Landrace, Norwegian Yorkshire, Ossabaw Island, Oxford Sandy and Black, Pakchong 5, Philippine Native, Pietrain, Poland China, Red Wattle, Saddleback, Semirechensk, Siberian Black Pied, Small Black, Small White, Spots, Surabaya Babi, Swabian-Hall, Swedish Landrace, Swallow Belied Mangalitza, Taihu pig, Tamworth, Thuoc Nhieu, Tibetan, Tokyo-X, Tsivilsk, Turopolje, Ukrainian Spotted Steppe, Ukrainian White Steppe, Urzhum, Vietnamese Potbelly, Welsh, Wessex Saddleback, West French White, Windsnyer, Wuzhishanm, Yanan, Yorkshire and Yorkshire Blue and White. In some embodiments, the porcine cells are Yorkshire and Yucatan porcine cells.

In some embodiments, cells of the present disclosure are islet cells or a subset thereof. The islet cells may comprise beta cells, alpha cells, delta cells, epsilon cells, or PP cells (aka gamma cells or F cells). In some embodiments, cells of the present disclosure are islets. In some embodiments, cells of the present disclosure are comprised in an intact pancreas. In some embodiments, cells of the present disclosure are comprised in a pancreas fragment. In some embodiments, cells of the present disclosure are comprised in a pancreatic organoid. In some embodiments, cells of the present disclosure are comprised in an aggregate of cells. In some embodiments, cells of the present disclosure are cells dispersed in a medium (e.g., a solid, a semi-solid, a gel, a liquid, or a combination thereof). In some embodiments, cells of the present disclosure are comprised in cell clusters. In some embodiments, islet cells or organoids of the present disclosure are substantially free of pancreatic exocrine cells.

In some embodiments, the cells, tissues, organs or animals of the present disclosure have been genetically modified such that one or more genes has been modified by addition, deletion, inactivation, disruption, excision of a portion thereof, or a portion of the gene sequence has been altered.

In some embodiments, the cells, tissues, or organs of the disclosure comprise one or more mutations that inactivate one or more genes. In some embodiments, the cells, tissues, organs or animals comprise one or more mutations or epigenetic changes that result in decreased or eliminated expression of one or more genes having the one or more mutations. In some embodiments, the one or more genes is inactivated by genetically modifying the nucleic acid(s) present in the cells, tissues, organs or animals. In some embodiments, the inactivation of one or more genes is confirmed by means of an assay. In some embodiments, the assay is a reverse transcriptase PCR assay, RNA-seq, real-time PCR, or junction PCR mapping assay. In some embodiments, the assay is an enzymatic assay for the function of the gene protein or an immunoassay for a protein transcribed from the gene or a fragment of the gene.

The cells, tissues, or organs of the present disclosure can be genetically modified by any suitable method. Non-limiting examples of suitable methods for the knockout (KO), knockin (KI), and/or genomic replacement strategies disclosed and described herein include CRISPR-mediated genetic modification using Cas9, Cas12a (Cpf1), Cas12b, Cas12c, Cas12 d, Cas12e, Cas12g, Cas12h, Cas12i, or other CRISPR endonucleases, Argonaute endonucleases, transcription activator-like (TAL) effector and nucleases (TALEN), zinc finger nucleases (ZFN), expression vectors, transposon systems (e.g., PiggyBac transposase), or any combination thereof. In some embodiments,

In some embodiments, the cells, tissues, or organs are substantially free of enzymatic activity of at least one glycosyltransferase enzyme, wherein said glycosyltransferase enzyme is GGTA, B4GALNT2, or CMAH. The cells, tissues, or organs can be substantially free of enzymatic activity of at least two glycosyltransferase enzymes selected from GGTA, B4GALNT2, and CMAH. The cells, tissues, or organs can be substantially free of enzymatic activity of three glycosyltransferase enzymes selected from GGTA, B4GALNT2, and CMAH. In some cases, the cells substantially free of enzymatic activity of at least one glycosyltransferase enzyme selected from GGTA, B4GALNT2, and CMAH are substantially free of detectable levels of a full-length copy of the glycosyltransferase enzyme protein. In some cases, the cells substantially free of enzymatic activity of at least one glycosyltransferase enzyme selected from GGTA, B4GALNT2, and CMAH are substantially free of detectable levels of a functional polypeptide fragment of the glycosyltransferase enzyme protein. In some cases, the cells substantially free of enzymatic activity of at least one glycosyltransferase enzyme selected from GGTA, B4GALNT2, and CMAH are substantially free of transcription of mRNA encoding the full-length glycosyltransferase enzyme. In some cases, the cells substantially free of enzymatic activity of at least one glycosyltransferase enzyme selected from GGTA, B4GALNT2, and CMAH are substantially free of transcription of mRNA encoding a functional fragment of the glycosyltransferase enzyme. In some cases, the cells substantially free of enzymatic activity of at least one glycosyltransferase enzyme selected from GGTA, B4GALNT2, and CMAH comprise an indel within an open reading frame of the at least one glycosyltransferase enzyme. The indel may be generated using site-directed nuclease. The indel may disrupt the open reading frame (ORF) (or in the case of a gene having multiple copies within the genome, all of the ORFs) of the at least one glycosyltransferase enzyme such that when the glycosyltransferase gene is transcribed, production of a full length or functional fragment mRNA or protein is prevented.

In some embodiments, the cells, tissues, or organs express at least two polypeptide sequences (e.g., at least two heterologous polypeptide sequences) derived from a non-porcine mammalian species, wherein said at least two polypeptide sequences comprise at least two of CD46, CD55, CD59, THBD, TFPI, CD39, B2M, HLAE, CD47, A20, PD-L1, FASL, or HO-1. The cells, tissues, or organs may express at least three, at least four, at least five, at least six, at least seven, at least eight, at least nine, at least ten, at least eleven, at least twelve, or all of CD46, CD55, CD59, THBD, TFPI, CD39, B2M, HLAE, CD47, A20, PD-L1, FASL, or HO-1. In some cases, the at least two polypeptide sequences derived from a non-porcine mammalian species comprise a full-length sequence of CD46, CD55, CD59, THBD, TFPI, CD39, B2M, HLAE, CD47, A20, PD-L1, FASL, or HO-1, or a combination thereof. In some cases, the at least two polypeptide sequences derived from a non-porcine mammalian species comprise a functional fragment of CD46, CD55, CD59, THBD, TFPI, CD39, B2M, HLAE, CD47, A20, PD-L1, FASL, or HO-1, or a combination thereof. In some cases, the cells, tissues, or organs expressing at least two polypeptide sequences derived from a non-porcine mammalian species express mRNA encoding a full-length sequence of CD46, CD55, CD59, THBD, TFPI, CD39, B2M, HLAE, CD47, A20, PD-L1, FASL, or HO-1, or a combination thereof. In some cases, the cells, tissues, or organs expressing at least two polypeptide sequences derived from a non-porcine mammalian species express mRNA encoding a functional fragment sequence of CD46, CD55, CD59, THBD, TFPI, CD39, B2M, HLAE, CD47, A20, PD-L1, FASL, or HO-1, or a combination thereof.

In some embodiments, any one of the heterologous polypeptide sequences disclosed herein is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a polypeptide sequence encoded by a human gene of interest or a fragment thereof. In some embodiments, a polynucleotide sequence encoding the any one of the heterologous polypeptide sequences disclosed herein is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a human gene of interest or a fragment thereof. In some examples, the human gene of interest disclosed herein may comprise one or more members (e.g., two or more members) selected from: CD46, CD55, CD59, THBD, TFPI, CD39, B2M, HLAE, CD47, A20, PD-L1, FASL, and HO-1.

In some embodiments, any of the genetically modified cells, tissues or organs disclosed herein may be used to treat a subject of a different species as the genetically modified cells. In some embodiments, the disclosure provides for methods of transplanting any of the genetically modified cells, tissues or organs described herein into a subject in need thereof. In some embodiments, the subject is a human. In some embodiments, the subject is a non-human primate.

The non-porcine mammalian species may be a primate species. In some embodiments, the non-porcine mammalian species is a non-human primate.

In some embodiments, the non-porcine mammalian species is Homo sapiens.

In some cases, the cells, tissues, or organs expressing at least two polypeptide sequences derived from a non-porcine mammalian species, comprise a genomic sequence encoding CD46, CD55, CD59, THBD, TFPI, CD39, B2M, HLAE, CD47, A20, PD-L1, FASL, or HO-1, a combination thereof, or a fusion thereof. In some cases, the genomic sequence comprises an open reading frame encoding a full-length copy of CD46, CD55, CD59, THBD, TFPI, CD39, B2M, HLAE, CD47, A20, PD-L1, FASL, or HO-1, a combination thereof, or a fusion thereof. In some cases, the genomic sequence comprises an open reading frame encoding a functional fragment of CD46, CD55, CD59, THBD, TFPI, CD39, B2M, HLAE, CD47, A20, PD-L1, FASL, or HO-1, a combination thereof, or a fusion thereof. In some embodiments, the open reading frame is operably linked to a promoter. In some embodiments, the promoter is a ubiquitous promoter. In some embodiments, the promoter is a human promoter. In some embodiments, the promoter is a non-porcine promoter. In some embodiments, the promoter is a viral promoter. In some embodiments, the promoter is a porcine promoter. In some embodiments, the promoter is a ubiquitous promoter. In some embodiments, the promoter is the natural human promoter or a functional fragment thereof of a gene derived from the non-porcine mammalian species, e.g. the natural promoter of CD46, CD55, CD59, THBD, TFPI, CD39, B2M, HLAE, CD47, A20, PD-L1, FASL, or HO-1. In some embodiments, the promoter is the porcine promoter or a functional fragment thereof of the porcine ortholog of a gene derived from the non-porcine mammalian species, e.g. the promoter of CD46, CD55, CD59, THBD, TFPI, CD39, B2M, HLAE, CD47, A20, PD-L1, FASL, or HO-1.

The genomic sequences encoding the at least two polypeptide sequences may be located at any suitable location in the genome of the cells, tissues, or organs. In some embodiments, the genomic sequences encoding the at least two polypeptide sequences are located at a “safe harbor” locus in the porcine genome such as AAVS1, CEP112, ROSA26, Pifs302, or Pifs501. In some embodiments, the genomic sequences encoding the at least two polypeptide sequences are located at or proximal to the locus of another gene that has been “knocked out” by indel formation using a site-directed or programmable nuclease (e.g. GGTA, B4GALNT2, CMAH mentioned above). In some embodiments, the genomic sequences encoding the at least two polypeptide sequences are located at the corresponding orthologous porcine locus for the polypeptide derived from the non-porcine mammalian species, e.g. the locus of an ortholog of CD46, CD55, CD59, THBD, TFPI, CD39, B2M, HLAE, CD47, A20, PD-L1, FASL, or HO-1. In some embodiments, the genomic sequences encoding the at least two polypeptide sequences are located in place of the corresponding orthologous porcine polypeptide, e.g. an ortholog of CD46, CD55, CD59, THBD, TFPI, CD39, B2M, HLAE, CD47, A20, PD-L1, FASL, or HO-1.

In some embodiments, at least two polypeptide sequences derived from a non-porcine mammalian species comprise a subset of CD46, CD55, CD59, THBD, TFPI, CD39, B2M, HLAE, CD47, A20, PD-L1, FASL, or HO-1. The subset may be CD46, CD55, CD59, CD39, B2M, HLAE, and CD47. The subset may be transgenes of at least two types selected from the group consisting of inflammatory response transgenes, immune response transgenes, immunomodulator transgenes, coagulation response transgenes, complement response transgenes, and combinations thereof. Inflammatory response transgenes may comprise TNF α-induced protein 3 (A20), heme oxygenase (HO-1), Cluster of Differentiation 47 (CD47), or combinations thereof. Immune response transgenes may comprise human leukocyte antigen-E (HLA-E), beta-2 microglobulin (B2M), or combinations thereof. Immunomodulator transgenes may comprise programmed death-ligand 1 (PD-L1), Fas ligand (FasL), or combinations thereof. Coagulation response transgenes may comprise Cluster of Differentiation 39 (CD39), thrombomodulin (THBD), tissue factor pathway inhibitor (TFPI), and combinations thereof. Complement response transgenes may comprise membrane cofactor protein (hCD46), complement decay accelerating factor (hCD55), MAC-inhibitor factor (hCD59), or combinations thereof.

In some embodiments, the at least two polypeptide sequences derived from a non-porcine mammalian species may be provided as a tandem sequence (e.g. as a single construct integrated e.g. by homologous recombination).

In some embodiments, the cells, tissues, or organs described herein may display survival greater than about 1 day, 2 days, 3 days, 4 days, 5 days, 6 days, 7 days, 8 days, 9 days, 10 days, 11 days, 12 days, 13 days, 14 days, 15 days, 16 days, 20 days, 24 days, 36 days, 48 days, 60 days, 72 days, 84 days, or more when transplanted into non-porcine mammalian species.

In some embodiments, the cells, tissues, or organs described herein may display an altered immune response when transplanted into a non-porcine mammalian species described herein. The cells, tissues, or organs described herein may display a reduced IBMIR to PBMCs isolated from a non-porcine mammalian species. The Instant Blood Mediated Immune Reaction (IBMIR) is the potent innate immune response, including coagulation and complement cascades and leukocyte and platelet populations, induced shortly after transplantation of donor islets to a recipient which can be measured, e.g., using assays to monitor complement activation (Kourtzelis et al., Chapter 11 “Regulation of Instant Blood Mediated Inflammatory Reaction (IBMIR) in Pancreatic Islet Xeno-Transplantation: Points for Therapeutic Interventions” in J. D. Lambris et al. (eds.), Immune Responses to Biosurfaces (2015), Advances in Experimental Medicine and Biology, Springer International). The IBMIR may be reduced by at least about 0.25-fold, about 0.5-fold, about 0.75-fold, about 1-fold, about 2-fold, about 3-fold, about 4-fold, about 5-fold, about 6-fold, about 7-fold, about 8-fold, about 9-fold, or about 10-fold, or more. The cells, tissues, or organs described herein may display reduced toxicity from complement derived from a non-porcine species. The toxicity from complement derived from non-porcine species may be measured using a radioactive assay (e.g., a ⁵¹Cr assay), live cell staining (e.g., by flow cytometry), the activity of released intracellular enzymes, such as LDH or GAPDH, or dead cell staining. An exemplary method is described in Yamamoto et al. Scientific Reports (10): 9771 (2020). The toxicity to complement derived from a non-porcine species may be reduced by at least about 0.25-fold, about 0.5-fold, about 0.75-fold, about 1-fold, about 2-fold, about 3-fold, about 4-fold, about 5-fold, about 6-fold, about 7-fold, about 8-fold, about 9-fold, or about 10-fold, or more. The cells, tissues, or organs described herein may display reduced induction of activated protein C coagulation derived from a non-porcine mammalian species. The induction of activated protein C coagulation derived from a non-porcine mammalian species may be reduced by at least about 0.25-fold, about 0.5-fold, about 0.75-fold, about 1-fold, about 2-fold, about 3-fold, about 4-fold, about 5-fold, about 6-fold, about 7-fold, about 8-fold, about 9-fold, or about 10-fold, or more. The cells, tissues, or organs described herein may display reduced induction of thrombin-antithrombin complex formation derived from a non-porcine species. The induction of thrombin-antithrombin complex formation derived from a non-porcine species may be reduced by at least about 0.25-fold, about 0.5-fold, about 0.75-fold, about 1-fold, about 2-fold, about 3-fold, about 4-fold, about 5-fold, about 6-fold, about 7-fold, about 8-fold, about 9-fold, or about 10-fold, or more. The cells, tissues, or organs described herein may display reduced toxicity from NK cells derived from a non-porcine species. The toxicity from NK cells derived from non-porcine species may be measured using a radioactive assay (e.g., a ⁵¹Cr assay), live cell staining (e.g., by flow cytometry), the activity of released intracellular enzymes, such as LDH or GAPDH, dead cell staining, or other techniques used for assessing cytotoxicity. The toxicity from NK cells derived from a non-porcine species may be reduced by at least about 0.25-fold, about 0.5-fold, about 0.75-fold, about 1-fold, about 2-fold, about 3-fold, about 4-fold, about 5-fold, about 6-fold, about 7-fold, about 8-fold, about 9-fold, or about 10-fold, or more.

In some cases, the present disclosure provides for a composition comprising a therapeutically effective dose of porcine islet cells according to any of the embodiments described herein. The islet cells may comprise islet cells in their natural proportions found in the pancreas, or may comprise a subset of islet cells, or islet cells at different proportions than naturally found in the pancreas. The islet cells may comprise beta cells, alpha cells, delta cells, epsilon cells, or PP cells (aka gamma cells or F cells).

In some cases, the islet cells may comprise beta cells at an amount of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 50%, or more. In some cases, the islet cells may comprise beta cells at an amount of at most about 50%, 45%, 40%, 35%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less. In some examples, the islet cells may comprise beta cells at an amount ranging from about 10% to about 30%. In some examples, the islet cells may comprise beta cells at an amount ranging from about 12% to about 25%.

In some cases, the islet cells may comprise alpha cells at an amount of at least about 1%, 2%, 3%, 4%, %, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 50%, or more. In some cases, the islet cells may comprise alpha cells at an amount of at most about 50%, 45%, 40%, 35%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less. In some examples, the islet cells may comprise alpha cells at an amount ranging from about 10% to about 40%. In some examples, the islet cells may comprise beta cells at an amount ranging from about 15% to about 30%.

In some cases, the islet cells may comprise delta cells at an amount of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 50%, or more. In some cases, the islet cells may comprise delta cells at an amount of at most about 50%, 45%, 40%, 35%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less. In some examples, the islet cells may comprise delta cells at an amount ranging from about 10% to about 40%. In some examples, the islet cells may comprise delta cells at an amount ranging from about 15% to about 30%.

In some cases, the islet cells may comprise epsilon cells at an amount of at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 50%, or more. In some cases, the islet cells may comprise epsilon cells at an amount of at most about 50%, 45%, 40%, 35%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less. In some examples, the islet cells may comprise epsilon cells at an amount ranging from about 10% to about 40%. In some examples, the islet cells may comprise epsilon cells at an amount ranging from about 15% to about 30%.

In some cases, the islet cells may comprise pancreatic polypeptide (PP) cells at an amount of at least about 1%, 2%, 3%, 4%, 5%, 6%, 7%, 8%, 9%, 10%, 1%, 2%, 13%, 14%, 15%, 16%, 17%, 18%, 19%, 20%, 21%, 22%, 23%, 24%, 25%, 26%, 27%, 28%, 29%, 30%, 35%, 40%, 50%, or more. In some cases, the islet cells may comprise PP cells at an amount of at most about 50%, 45%, 40%, 35%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, or less. In some examples, the islet cells may comprise PP cells at an amount ranging from about 10% to about 40%. In some examples, the islet cells may comprise PP cells at an amount ranging from about 15% to about 30%.

In some cases, a number of beta cells as compared to a number of alpha cells may be at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, or more. In some cases, a number of beta cells as compared to a number of alpha cells may be at most about 50%, 45%, 40%, 35%, 30%, 29%, 28%, 27%, 26%, 25%, 24%, 23%, 22%, 21%, 20%, 19%, 18%, 17%, 16%, 15%, 14%, 13%, 12%, 11, 1%, 9%, 8%, 7%, 6%, 5%, 4%, 3% 2%, 1%, or less. In some examples, a number of beta cells as compared to a number of alpha cells may range between about 10% to about 40%. In some examples, a number of beta cells as compared to a number of alpha cells may range between about 15% to about 30%.

The cells may be formulated by first harvesting them from their culture medium or from a disaggregated pancreas, and then washing and concentrating the cells in a medium and container system suitable for administration (a “pharmaceutically acceptable” carrier) in a treatment-effective amount. Suitable infusion medium can be any isotonic medium formulation such as normal saline, Normosol R (Abbott), Plasma-Lyte A (Baxter), 5% dextrose in water, Ringer's lactate, CMIRL 1066 without phenol red plus Heparin (e.g., 100 U/kg recipient), etc. can be utilized. The infusion medium can be supplemented with human serum albumin, fetal bovine serum or other human serum components. In some cases, an anti-coagulant (e.g., heparin) may be administered at an amount of at least 1 unit per kilogram of recipient (U/kg), 2 U/kg, 3 U/kg, 4 U/kg, 5 U/kg, 10 U/kg, 15 U/kg, 20 U/kg, 30 U/kg, 40 U/kg, 50 U/kg, 60 U/kg, 70 U/kg, 80 U/kg, 90 U/kg, 100 U/kg, 150 U/kg, 200 U/kg, 250 U/kg, 300 U/kg, 350 U/kg, 400 U/kg, 450 U/kg, 500 U/kg, 600 U/kg, 700 U/kg, 800 U/kg, 900 U/kg, 1,000 U/kg, or more. The anti-coagulant may be administered in the same solution (e.g., buffer) as the islet cells. In other embodiments, the anti-coagulant and the islet cells may be administered separately. In some cases, a TNF-alpha inhibitor (e.g., Etanercept) may be administered at an amount of at least about 0.1 milligram per kilogram of recipient (mg/kg), 0.2 mg/kg, 0.3 mg/kg, 0.4 mg/kg, 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or more. In an example, the TNF-alpha inhibitor may be administered at an amount of about 3 mg/kg. The TNF-alpha inhibitor may be administered in the same solution (e.g., buffer) as the islet cells. In other embodiments, the TNF-alpha inhibitor and the islet cells may be administered separately.

In some cases, the present disclosure provides for a method of treating an insulin resistant or deficient condition in a non-porcine mammal in need thereof. The insulin resistant or deficient condition may comprise comprises type 1 or type 2 diabetes mellitus, monogenic diabetes syndromes (such as neonatal diabetes and maturity-onset diabetes of the young [MODY]), diseases of the exocrine pancreas (such as cystic fibrosis and pancreatitis), or drug- or chemical-induced diabetes (such as with glucocorticoid use, in the treatment of HIV/AIDS, or after organ transplantation). In some embodiments when the insulin resistant or deficient condition comprises type 1 or type 2 diabetes mellitus, the mammal may exhibit particular clinical criteria such as fasting plasma glucose levels, performance on an oral glucose tolerance test, or HbA1C. In some embodiments, the insulin resistant or deficient condition may comprise enhanced risk factors present in the mammal such as unstable diabetes, hypoglycemia unawareness, severe hypoglycemic episodes, or glycemic lability.

The non-porcine mammalian species may be a primate species. In some embodiments, the non-porcine mammalian species is a non-human primate. The non-human primate includes non-human living primates according to any or all of various classifications of non-human living primates, including, but not limited to, families Callitrichidae (marmosets and tamarins), Cebidae (New World monkeys), Cercopithecidae (Old World monkeys), Cheirogaleidae (dwarf lemurs and mouse lemurs), Daubentoniidae (aye-aye), Galagonidae (bushbabies and galagos), Hominidae (including great apes), Hylobatidae (gibbons and lesser apes), Indridae (indris, sifakas, and relatives), Lemuridae (true lemurs), Loridae (lorises), Megaladapidae (sportive lemurs), and Tarsiidae (tarsiers). The term “non-human primates” encompasses non-human primates and groups thereof classified according to any or all of various classifications of non-human living primates. For example, Wilson and Reeder (1993) split Megaladapidae from Lemuridae, Galagonidae from Loridae (and in spelling the latter Loridae rather than Lorisidae), and include the great apes in Hominidae. Wilson, D. E., and D. M. Reeder. 1993. Mammal Species of the World, A Taxonomic and Geographic Reference. 2nd edition. Smithsonian Institution Press, Washington. Anderson and Jones (1984) divide the order of living primates (Primates) into two suborders, the Strepsirhini and the Haplorhini. Thorington, R. W., Jr., and S. Anderson. 1984. Primates. Pp. 187-217 in Anderson, S. and J. K. Jones, Jr. (eds). Orders and Families of Recent Mammals of the World. John Wiley and Sons, N.Y. The Strepsirhines include mostly arboreal species with many primitive characteristics, but at the same time, some extreme specializations for particular modes of life, and wherein the Haplorhines are the so-called “higher” primates, further divided into two major groups, the Platyrrhini and the Catarrhini. Platyrrhines have flat noses, outwardly directed nasal openings, three premolars in upper and lower jaws, anterior upper molars with 3 or 4 major cusps, and are found only in the New World (families Cebidae and Callitrichidae). Catarrhines have paired downwardly directed nasal openings, which are close together; usually two premolars in each jaw, anterior upper molars with 4 cusps, and are found only in the Old World (Cercopithecidae, Hylobatidae, Hominidae). Most primate species live in the tropics or subtropics, although a few also inhabit temperate regions. Except for a few terrestrial species, primates are arboreal. Some species eat leaves or fruit; others are insectivorous or carnivorous. See Myers, P. 1999. “Primates” (On-line), Animal Diversity Web. Accessed Aug. 26, 2005.

In some embodiments, the non-porcine mammalian species is Homo sapiens.

The method of treating the insulin resistant or deficient condition in the non-porcine mammal in need thereof may involve the administration or transplant of any of the compositions, cells, organs, or tissues described herein. In some cases, when a cell composition is administered, the composition is centrally administered, e.g. is administered via an internal jugular vein or a hepatic portal vein of the non-porcine mammal.

In some cases, prior to treatment with the compositions, cells, organs, or tissues, the non-porcine mammal has received an induction immunosuppression regimen. The induction regimen may comprise therapeutically effective doses of anti-thymocyte globulin, anti-CD40 antibody, anti-CD20 antibody, a rapalog, a calcineurin inhibitor, ganciclovir or a prodrug thereof, an antihistamine, or a corticosteroid prior to administering said cell, tissue, or organ.

In some cases, after treatment with the compositions, cells, organs, or tissues, the non-porcine mammal receives a maintenance immunosuppression regimen. The maintenance regimen may comprise therapeutically effective doses of anti-CD40 antibody, a rapalog, a calcineurin inhibitor, and ganciclovir or a prodrug of ganciclovir.

In some cases, after treatment with the compositions, cells, organs, or tissues, the non-porcine mammal receives a supportive insulin regimen. The supportive insulin regimen may comprise therapeutically effective doses of an intermediate- or long-acting insulin analog (e.g. insulin glargine, insulin detemir, or NPH insulin) following administration of said cells, tissue, organ, or composition.

The method of treating the insulin resistant or deficient condition in the non-porcine mammal in need thereof may involve the administration of a particular islet equivalent dose (IEQ per kg). For reference, islet equivalency (IEQ) is defined as one IEQ equaling a single spherical islet of 150 m in diameter (Huang et al. Cell Transplant 2018 July: 27(7): 1017-26), and islet equivalent dose is the IEQ per kg of the recipient non-porcine mammal body weight. In some cases, the dose may be at least about 1,000 IEQ per kg of non-porcine mammal body weight (IEQ/kg), 2,000 IEQ/kg, 3,000 IEQ/kg, 4,000 IEQ/kg, 5,000 IEQ/kg, 6,000 IEQ/kg, 7,000 IEQ/kg, 8,000 IEQ/kg, 9,000 IEQ/kg, 10,000 IEQ/kg, 11,000 IEQ/kg, 12,000 IEQ/kg, 13,000 IEQ/kg, 14,000 IEQ/kg, 15,000 IEQ/kg, 16,000 IEQ/kg, 17,000 IEQ/kg, 18,000 IEQ/kg, 19,000 IEQ/kg, 20,000 IEQ/kg, or more. In some cases, the dose may be at most about 20,000 IEQ/kg, 19,000 IEQ/kg, 18,000 IEQ/kg, 17,000 IEQ/kg, 16,000 IEQ/kg, 15,000 IEQ/kg, 14,000 IEQ/kg, 13,000 IEQ/kg, 12,000 IEQ/kg, 11,000 IEQ/kg, 10,000 IEQ/kg, 9,000 IEQ/kg, 8,000 IEQ/kg, 7,000 IEQ/kg, 6,000 IEQ/kg, 5,000 IEQ/kg, 4,000 IEQ/kg, 3,000 IEQ/kg, 2,000 IEQ/kg, 1,000 IEQ/kg, or less. In an example, the dose may be at least 5,000 IEQ per kg of non-porcine mammal body weight.

In some aspects, the present disclosure provides for a method of improving yield of islets from a porcine donor prior to transplantation to a non-porcine mammalian recipient. The method may comprise providing pancreatic organoids, culturing said organoids in the presence of an effective concentration of a caspase inhibitor, and continuing culture in the presence of an effective concentration of a corticosteroid. The organoids may be cultured in the presence of caspase inhibitor for at least 30 minutes, at least 60 minutes, at least 90 minutes, at least 2 hours, at least 4 hours, at least 6 hours, at least 12 hours, at least 18 hours, at least 24 hours, at least 30 hours, at least 90 hours, at least 120 hours, at least 180 hours, at least 360 hours, at least 720 hours, or more. The organoids may be cultured in the presence of corticosteroid for at least 1, at least 2, at least 3, at least 4, at least 5, at least 6, at least 7, at least 8, at least 9, at least 10, at least 11, at least 12, at least 13, or at least 14 days following treatment with the caspase inhibitor. The pancreatic organoids may be isolated from a porcine animal on day 7 or earlier. The caspase inhibitor may be Z-VAD-FMK, Z-LEHD-FMK, Z-IETD-FMK, Emricasan, Z-VEIDFMK, Z-DEVD-CMK, MX1122, M867, MMPSI, an isatin sulfonamide, Boc-Asp-FMK, VX-166, Q-VD-OPh, or IDN-6556. The corticosteroid may be methylprednisolone. The organoids may be cultured in the presence of nicotinamide or a metabolically acceptable analog thereof.

### EXAMPLES

### Example 1—Construction and Characterization of Transgenic Porcine Animals and Endothelial Cells Derived Therefrom

CRISPR-Cas9 mediated NHEJ was used to functionally knock out the three major carbohydrate-producing glycosyltransferase/glycosylhydrolase genes GGTA1, CMAH, and B4GALNT2 in pig primary fibroblasts from Bama minipigs. Twelve human transgenes (CD46, CD55, CD59, CD39, CD47, A20, PD-L1, HLA-E, B2M, THBD, TFPI, HO-1) were then integrated into a single multi-transgene cassette in the pig genome via PiggyBAC transposon-mediated random integration to generate 3KO/12TG cells designated “4-7”, which were used to generate pigs via somatic-cell nuclear transfer (SCNT). Wild-type porcine ear fibroblasts were first electroporated with both: a) CRISPR-Cas9 reagents targeting the GGTA, CMAH, and B4GALNT2 genes; and b) payload plasmids bearing (i) a PiggyBac transposase cassette (ii) a transgenic construct comprising one or more of the 12 human transgenes. The transgenes were arranged into 4 different cistrons with desired ubiquitous or tissue-specific promoters. The transgenes within each cistron were separated with ribosomal skipping 2A peptides to ensure expression in a similar molar ratio. Furthermore, a combination of cis-elements such as ubiquitous chromatin opening elements (UCOEs) were introduced to prevent transgene silencing and insulators with strong polyadenylation sites and terminators to minimize the interaction among transgenes and between transgenes and the flanking chromosome. Single-cell clones of the fibroblasts were generated and screened by fragment analysis/whole genome sequencing to identify clones with the desired genomic modifications, and a clone bearing the desired modifications was then used as a donor to produce a live pig by SCNT.

Transgene expression levels were determined by qPCR, integration site was determined using junction capture based on inverted PCR, and protein levels were determined via fluorescent-activated cell sorting (FACS). The results are summarized in Table 1B below.

TABLE 1A
DNA/mRNA/Protein Expression of knockout genes
and transgenes in 4-7 endothelial cells

IF KO/TG IN CELL LEVEL

| KO/TG | DNA | mRNA (full-length) | Protein (FACS) |
|---|---|---|---|
| GGTA (KO) | ✓ | NA | ✓ |
| CMAH (KO) | ✓ | NA | ✓ |
| B4Gal (KO) | ✓ | NA | NA |
| CD46 | ✓ | ✓ | ✓ |
| CD55 | ✓ | ✓ | ✓ |
| CD59 | ✓ | ✓ | ✓ |
| THBD | ✓ | None detected | None detected |
| TFPI | ✓ | ✓ | NA |
| CD39 | ✓ | ✓ | ✓ (high) |
| B2M-HLAE | ✓ | ✓ | ✓ (HLAE) |
| CD47 | ✓ | ✓ | ✓ |
| A20 | ✓ | ✓ Very low | None detected |
| PDL1 | ✓ | ✓ low | None detected |
| FasL | ✓ | ✓ low | None detected |

Table 1B shows expression of various transgenes in the tissues of 4-7 pigs by immunohistochemistry (IHC) staining results.

TABLE 1B
IHC Staining for Transgenes on 4-7 endothelial cells

| Target | 4-7 pig | Human | WT pig |
|---|---|---|---|
| CD46 | + | ++ | − |
| CD55 | ++ | +++ | − |
| CD59 | + | +++ | − |
| B2M | +/− | ++ | − |
| TFPI | +/− | ++ | − |
| CD39 | + | ++ | − |
| HO-1 | +++ | ++ | + |
| A20 | − | ++ | − |
| EPCR | − | ++ | − |
| CD47 | + | +++ | − |
| HLA-E | + | + | − |
| GGTA | − | − | +++ |

Functional Characterization of Consequences of Gene Knockouts/Knockins

GGTA/CMAH/B4Gal

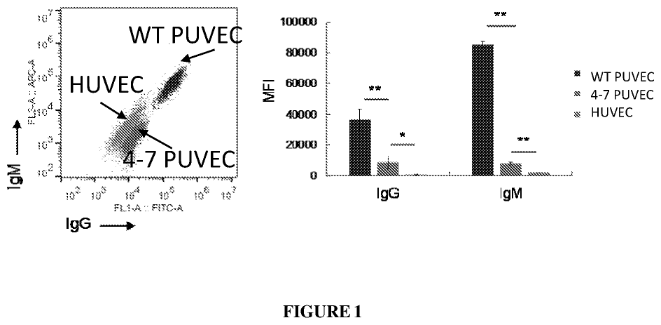

Preformed antibodies that bind wild-type pig tissue have been considered a major initial immunologic barrier to xenotransplantation, and these three genes have been identified as being largely responsible for producing the xenogenic antigens targeted by these antibodies (Byrne 2014, Lai 2002, Lutz 2013, Martens 2017, Tseng 2006). Thus, it was predicted that the functional loss of these genes would largely eliminate the binding of preformed anti-pig antibodies to the endothelium of the porcine graft. This was confirmed by flow cytometry results showing decreased binding of host antibodies to target porcine umbilical vein endothelial cells (designated “4-7”, containing the GGTA1, CMAH, B4GalNT2 knockout, see FIG. 1). To demonstrate diminished antibody binding, genetically engineered pig endothelial cells were incubated with pooled human serum, and bound human IgM and IgG were detected with conjugated secondary anti-human antibodies and analyzed by flow cytometry. In contrast to wild-type pig umbilical vein endothelial cells (PUVEC) (red contour plot), elimination of the three genes resulted in a significant reduction in antibody binding (compare blue and yellow contour plots, ˜1 log decrease in binding).

Complement Regulatory Proteins (CD46, CD55, and CD59)

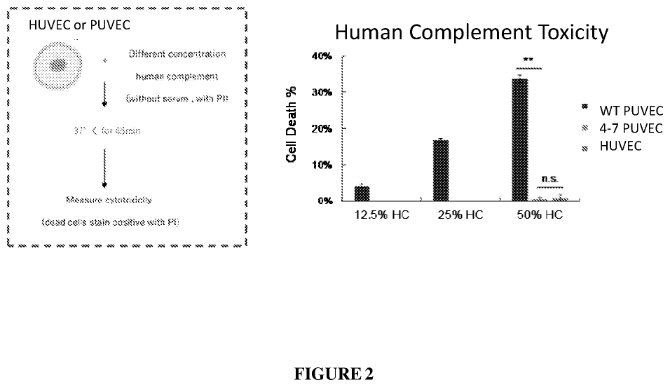

To maintain pig graft function and protect the donor organ from complement-mediated toxicity, human complement regulatory proteins were over-expressed. Briefly, genetically engineered pig fibroblasts and pig splenocytes were incubated with 25% human complement for one hour. Cells were stained with propidium iodide and analyzed by flow cytometry to quantify cell death (see FIG. 2). The left panel of FIG. 2 is a diagram illustrating the assay workflow, whereas the right panel is a chart illustrating the death of either human umbilical vein endothelial cells (“HUVEC”), transgenic 4-7 porcine umbilical vein endothelial cells (“4-7 PUVEC”), or normal porcine umbilical vein endothelial cells (“WT PUVEC”) after incubation with various concentrations of human complement (“HC”). 4-7 cells bearing all three transgenes show dramatically decreased death in response to human complement versus their normal pig counterparts, similar to human HUVEC cells.

Coagulation Response Genes

When vascularized WT porcine organs are transplanted into humans, preformed antibodies, complement, and innate immune cells can induce endothelial cell activation and trigger coagulation and inflammation. The incompatibility between coagulation regulatory factors from pig endothelial cells and human blood leads to abnormal platelet activation and thrombin formation, exacerbating the damage. In addition, molecular incompatibilities of coagulation regulators (e.g., tissue factor pathway inhibitor, TFPI) between pig and human render the extrinsic coagulation regulation ineffective.

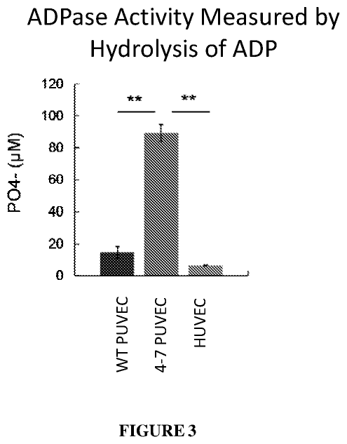

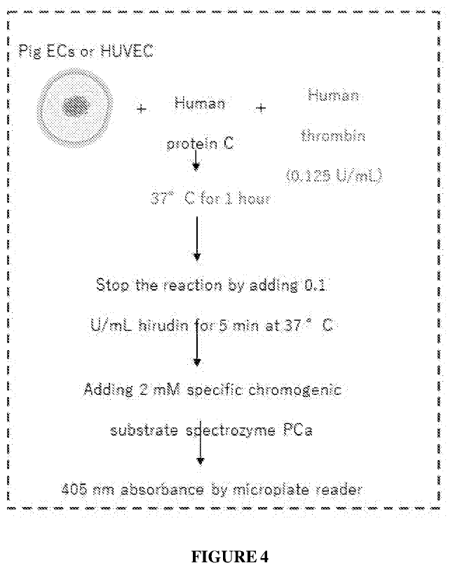

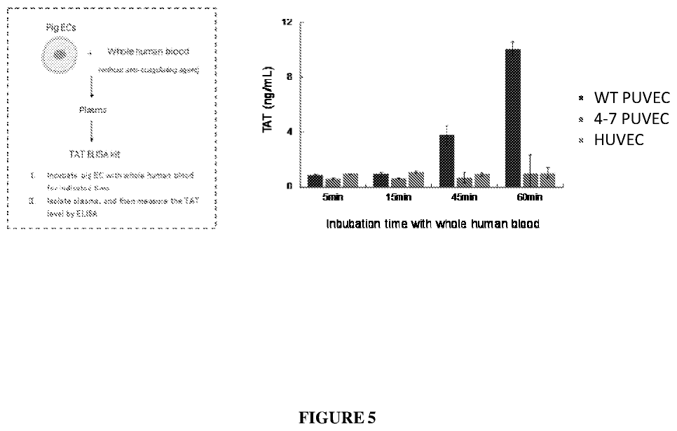

To address these xenogeneic coagulation issues, we overexpressed both: a) human CD39 (an ADP hydrolase that counteracts the thrombotic effect of ADP in the coagulation cascade) and b) human TFPI (a factor that translocates to the cell surface following endothelial cell activation) in the 4-7 PUVEC and then performed a variety of in vitro and ex vivo assays to validate the ability of these transgenes to function correctly and modulate platelet and coagulation cascades. FIG. 3 depicts results of analyses performed to validate expression/functionality of CD39 in 4-7 transgenic endothelial umbilical vein porcine cells (PUVECs). The chart shows the results of a colorimetric CD39 ADP-hydrolysis based activity assay performed on HUVECs, 4-7 PUVECs, or WT PUVECs; 4-7 cells show enhanced activity of CD39, suggesting the transgene is functional and overexpressed. In vitro ADPase biochemical assays showed significantly higher CD39 activity in 4-7 PUVECs vs WT PUVECs or HUVECs. Similarly, activated 4-7 PUVECs showed ability to effectively bind and neutralize human Xa, which can mitigate coagulation and reduce the formation of thrombin-antithrombin (TAT) complex (FIG. 5). The ex vivo coagulation assays in FIG. 5 with human whole blood co-cultured with 4-7 PUVECs demonstrate that minimal TAT (thrombin antithrombin) was formed, and the level of TAT formation was similar to that of HUVECs (FIG. 5), suggesting that 4-7 PUVECs gain enhanced coagulation compatibility with human factors.

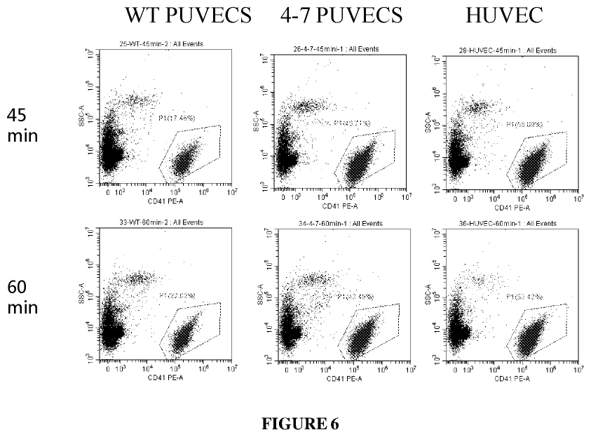

FIG. 6 shows results of assays done to evaluate effects of these genetic modifications on platelet activation. FIG. 6 depicts results of a platelet lysis assay performed on 4-7 transgenic cells. Shown are FACS traces quantitating the number of platelets remaining (outlined cluster) from human blood after incubation with HUVECs, 4-7 PUVECs, or WT PUVECs for 45 or 60 minutes. 4-7 cells continue to show elevated numbers of platelets remaining relative to porcine WT ECs, which is comparable to the fraction of platelets remaining when incubated with HUVEC cells.

HLA Components (HLA-E/B2M)

Ligation of MHC I on target cells with Killer Inhibitory Receptors (KIR) on natural killer (NK) cells inhibits NK cell-mediated killing of target cells. Pig MHC I is incapable of transmitting signals through the human NK KIR and thus pig cells are susceptible to targeted cell killing by NK cells. To overcome NK-mediated cell death, human HLA-E, which ligates human NK KIR receptors, was overexpressed in pig cells. Additionally, a human copy of the MHC heterodimerization partner B2M was also overexpressed.

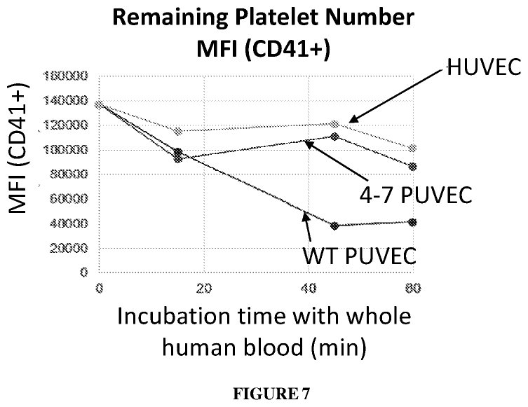

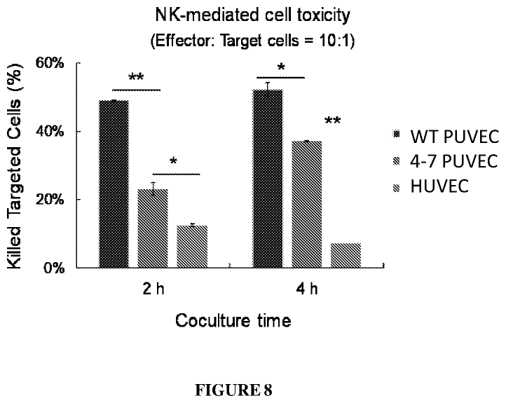

Functional assays were then performed to validate that 4-7 endothelial cells were resistant to NK-mediated cell killing due to the genetic modifications. WT PUVECs, 4-7 PUVECs, and HUVECs were targeted for killing by human NK cells in an in vitro assay (FIG. 7). FIG. 7 depicts that 4-7 PUVECs reveal significantly lower NK-mediated cytotoxicity than their WT counterpart (unpaired, two-tailed Student's t-test).

### Example 2—Isolation of Islets from Transgenic Animals

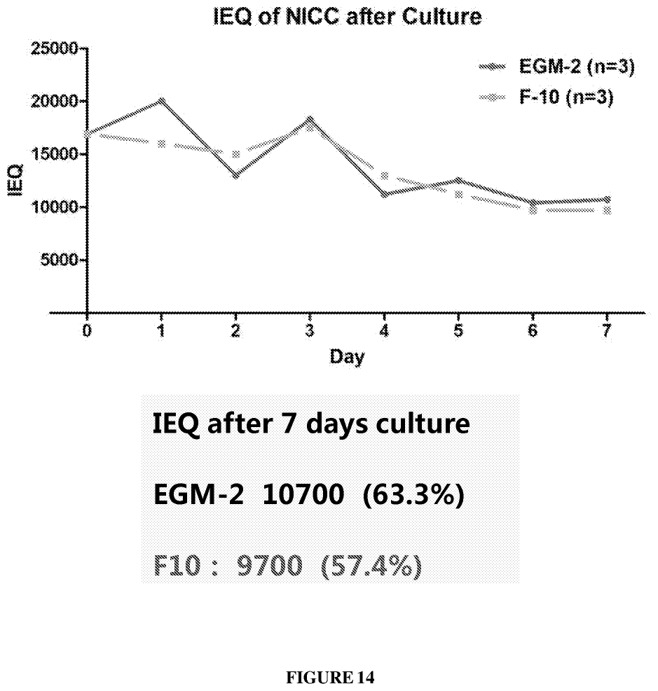

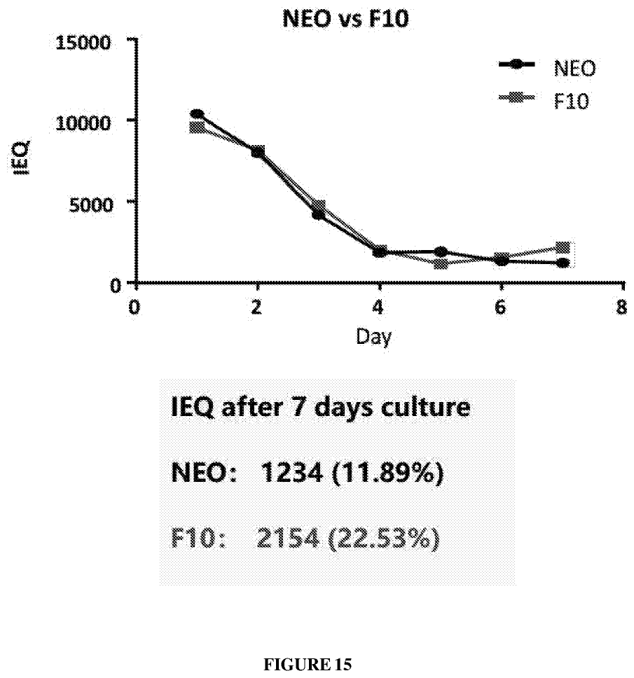

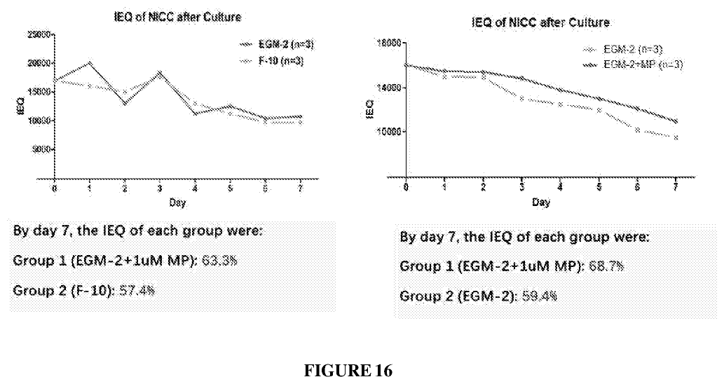

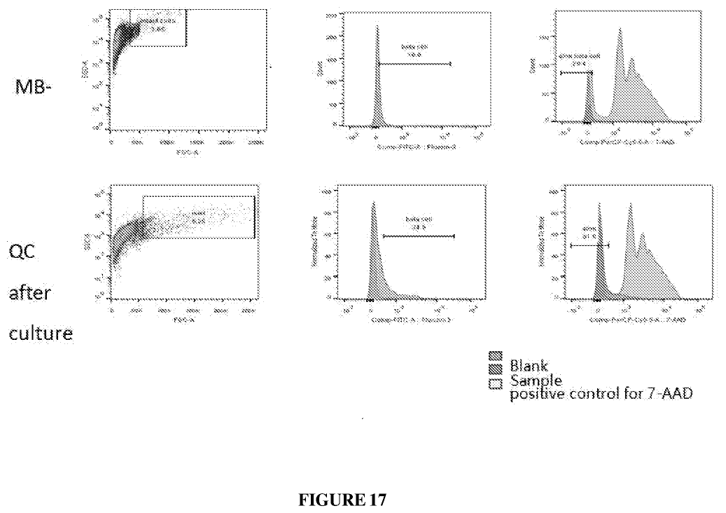

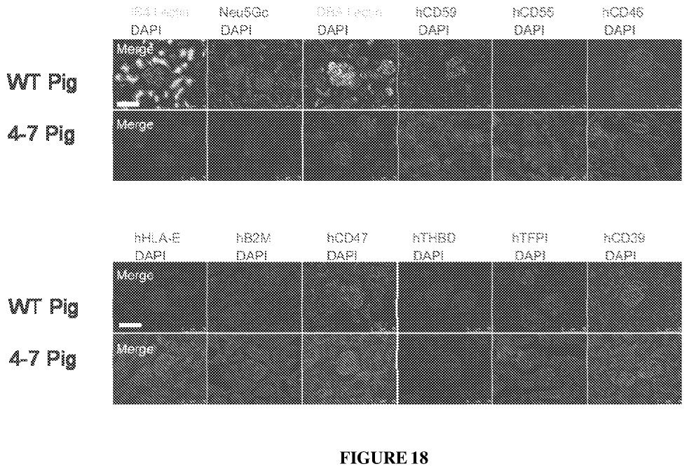

Transgenic male Bama minipigs (produced as in Example 1) were anesthetized and subjects to laparotomy and exsanguination at 0-7 days post birth. Pancreases were excised and were cut into small fragments under sterile conditions with a scalpel. Pancreatic fragments were subjected to collagenase V digestion (1 mg/ml) and transferred to a gas-permeable culture bag (OriGen PermaLife™ Cell Culture bags) and held at 22-24 dC for transport to the culture lab. Islets were cultured in bags or petri dishes in either EGM-2 medium (EGM-2 with FGF-B, VEGF, R3-IGF, ascorbic acid, hEGF, heparin, D-glucose, nicotinamide, 10% porcine serum, 50 μM IBMX, 120 μM amikacine, and 60 μM ampicillin), EGM-2 medium plus corticosteroid (EGM-2 plus 1 μM methylprednisolone), or Ham's F-10 medium for 7 days. Islet equivalents (IEQ) were measured over the 7 days in culture and graphed (see FIGS. 14, 15, and 16). EGM-2 medium with corticosteroid was associated with improved yields of islets among the 3 conditions.

In some cases, subsequent to mechanical or enzymatic digestion, pancreatic fragments may be purified by sedimentation (e.g., ficoll gradient sedimentation). In other cases, such purification step via sedimentation may not or need not be required. In such cases, the pancreatic fragments may be cultured (e.g., in culture dishes for about 7 days) before transplantation, during which time non-islet cells (e.g., exocrine cells) may die off.

### Example 3. Analysis of Islets from Transgenic Animals

Following isolation of islet cells from normal and transgenic Bama minipigs as in Example 2, assays were performed to evaluate the xenocompatibility aspects of 4-7 islet cells, similar to the scheme of experiments done in Example 1 on 4-7 endothelial cells.

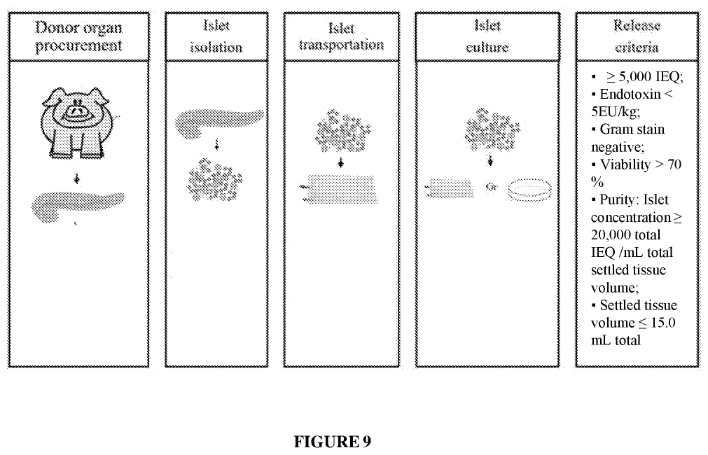

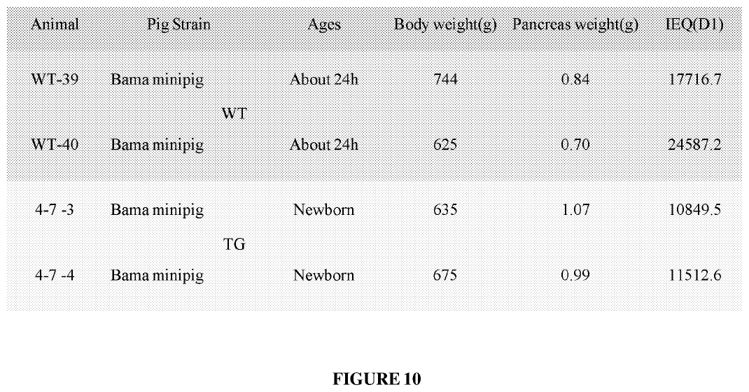

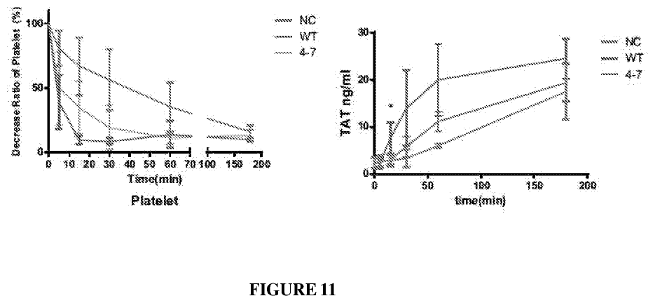

First, experiments were performed to evaluate the effect of the genetic modifications to modulate platelet and coagulation cascades. FIG. 11 (FIG. 11) depicts results of platelet lysis or TAT complex formation assays performed on islet cells isolated as in FIG. 9/Example 2. Shown are charts depicting platelet lysis assays as in FIG. 6 performed on human umbilical vein endothelial cells (HUVEC) as a negative control (NC), WT, or 4-7 islets (left panel) and TAT complex formation assays as in FIG. 5 performed on the HUVEC NC, WT, or 4-7 islets. 4-7 islets show decreased platelet lysis and reduced TAT complex formation when incubated with human blood components.

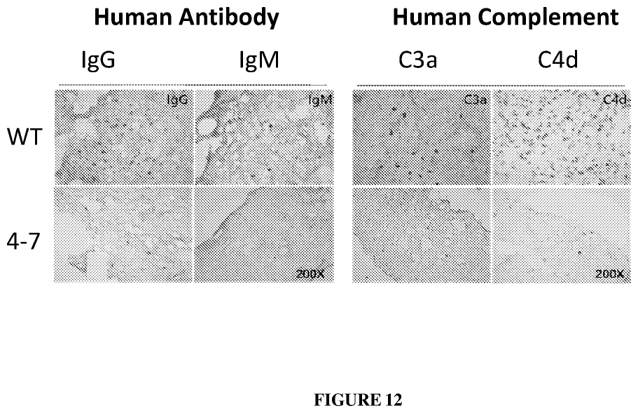

Second, experiments were performed to evaluate the effect of the genetic modifications to modulate the short-term IBMIR response of the human immune system to transplanted porcine tissue. FIG. 12 depicts results of instant blood-mediated inflammatory reaction (IBMIR) assays performed with human blood on 4-7 islets derived as in FIG. 9. Briefly, human whole blood was incubated with porcine islet cells as disclosed herein, and subsequently checked for coagulation or clotting that is caused at least in part by the contact (or interaction) between the human whole blood and the porcine islet cells. In some cases, the clot size and/or weight was measured. Shown in FIG. 9 are IHC micrographs at 200× magnification showing staining for antibody (IgG and IgM, left panel) and complement (C3a and C4 d, right panel) foci after incubation of 4-7 islet sections with human blood. 4-7 islet cells show decreased staining and foci associated with IgG, IgM, C3a, and C4 d, indicating the islet cells show reduced IBMIR and should show enhanced resistance to death upon initial transplantation.

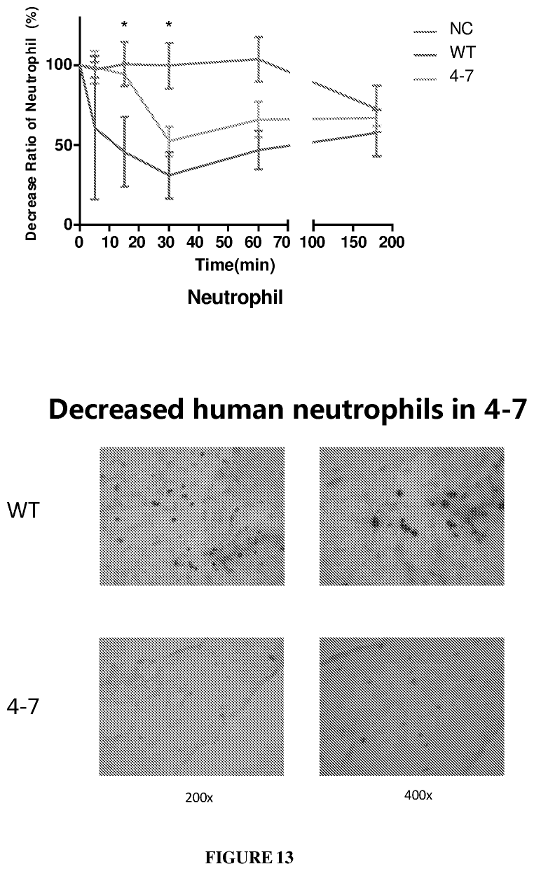

Third, experiments were performed to evaluate the effect of the genetic modifications to modulate activity of human neutrophils. FIG. 13 depicts the remaining numbers of neutrophil in the whole human blood incubated with 4-7 islets. 4-7 islets revealed higher remaining numbers of neutrophil compared to the WT islets when incubated with whole human blood

### Example 4—Transplantation of Islets into Recipient Mice

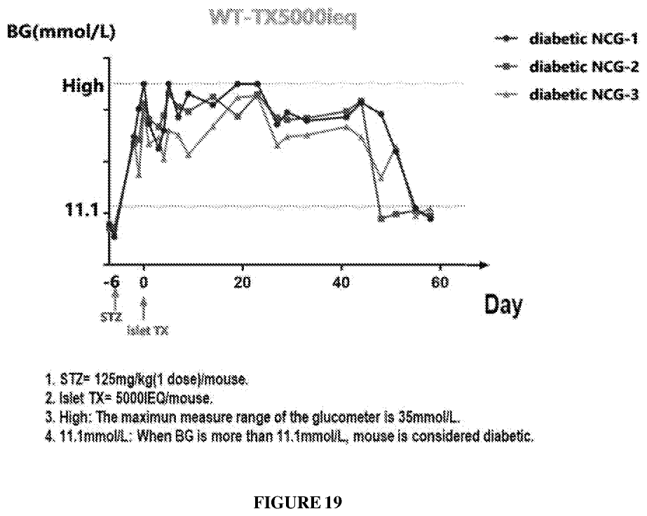

To test the functionality of the 4-7 porcine transgenic islet cells upon xenotransplantation, an STZ-based mouse diabetes islet adoptive transfer model was established. Exemplary blood glucose for mice using this model procedure is depicted in FIG. 19. This model uses a toxin (streptozotocin, STZ) to kill islet cells in immunodeficient mice, causing dramatic increases in blood glucose levels. Transplantation of islet cells results in normalization of blood glucose levels by ˜60 days post-transplant.

For assessing 4-7 islet cell efficacy in treating diabetes, diabetes was first induced in n=12 NCD (NOD-Prkdc^{em26cd52}Il2rg^{em26cd22}/NjuCrl) mice by treatment with a single dose of streptozotocin (STZ, 125 mg/kg) followed by a 3-day washout period, leaving 3 untreated age-matched mice as a control. The untreated mice and 3 of the STZ treated mice were then subjected to a sham transplantation operation, whereas 3 of the STZ mice received wild-type porcine islets (3000IEQ) isolated as in FIG. 9/Example 2 and 3 of the STZ mice received 4-7 transgenic porcine islets (3000IEQ) isolated as in FIG. 9/Example 2. Where islets were transplanted, they were transplanted under the left kidney capsule. Briefly, the 4-7 islet cells were mixed or dispersed in a solution (e.g., a buffer) and injected (e.g., slowly injected) under the kidney capsule via a syringe and soft tubes.

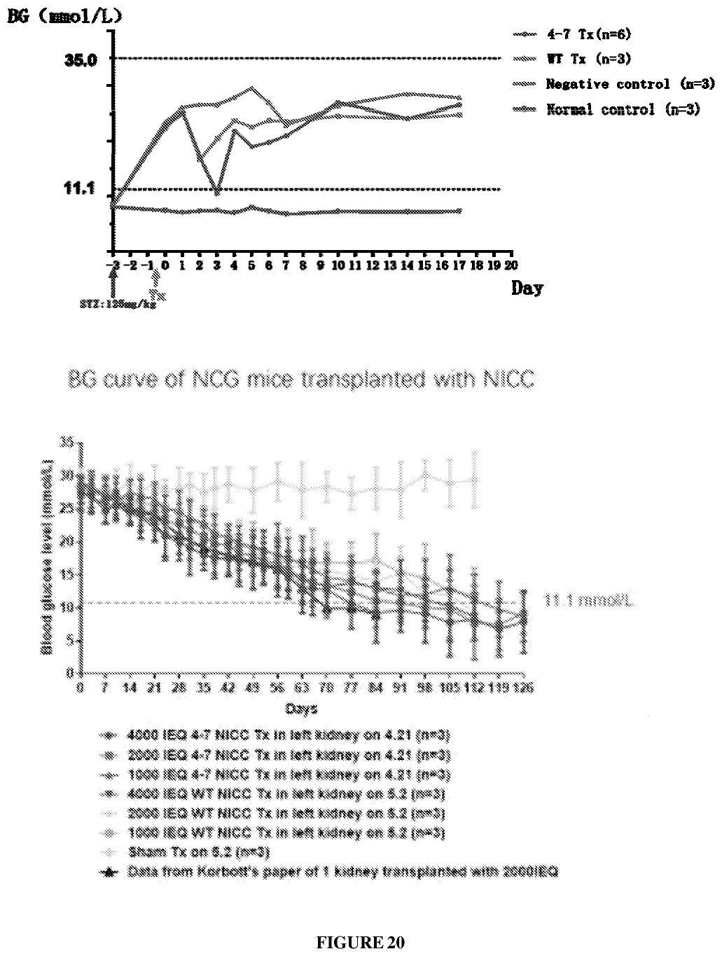

FIG. 20 shows blood glucose of NCG mice (as a T1D rodent model) receiving islet-like cell clusters (NICC) comprising the subject 4-7 porcine transgenic islet cells provided herein. NICC comprising wild-type pig islet cells were used as a control. Various amounts of NICC were transplanted to the NCG mice: 4000 IEQ, 2000 IEQ, and 1000 IEQ. Data indicates that the 4-7 porcine transgenic islet cells exhibited a similar efficacy in controlling the increased blood glucose level in mice, as compared to WT pig islet cells. The 4-7 porcine transgenic islet cells became functional (e.g., in controlling blood glucose level) in vivo at about two weeks after transplantation.

In some cases, abnormal growth of transplanted porcine cells may be monitored for a longer time (e.g., longer than 5, 6, 7, 8, 9, 10, 11, 12 months, or longer). In some cases, human adult islet cells may be used as a positive control, e.g., at a clinical human adult islet treatment dose. In some cases, non-obese diabetic (NOD) T1D mice model may be used as a secondary in vivo model. In some cases, porcine transgenic islet cells may be administered via intraportal vein injection to test its compatibility and safety.

### Example 5—Transplantation of Islets into Recipient Monkeys

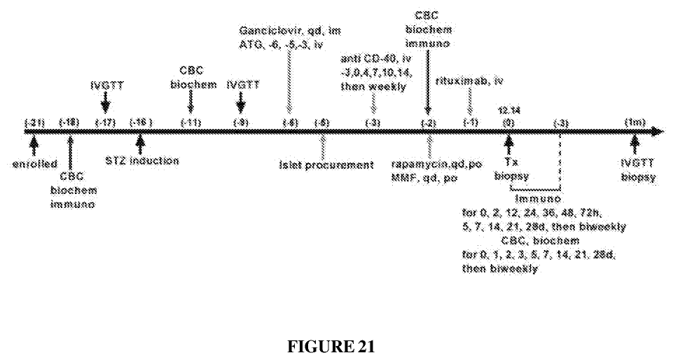

To test the functionality of the 4-7 porcine transgenic islet cells upon xenotransplantation to primates, an STZ-based NHP diabetes islet adoptive transfer (using intraportal islet cell transplantation) model was established. 4-7 islets and WT isolated as in FIG. 9/Example 2 were transplanted to Cynomolgus monkeys via percutaneous transhepatic portal catheterization guided by ultrasound. A scheme for these experiments is presented in FIG. 21.

The immunosuppression protocol used for transplant of porcine cells was as follows:

ATG was given IV on days −7 d (±2 d), −6 d (±2 d), −4 d (±2 d) at a dose of 5 mg/kg, and an additional dose of ATG was administered on ˜1 d if lymphocyte depletion to <5% of baseline level in the blood was achieved.

Anti-CD40 was given IV on −4 d (±1 d), 0 d, 4 d, 7 d, 10 d, 14 d and then weekly at a first dose of 50 mg/kg and 30 mg/kg then after.

Anti-CD20 monoclonal antibody Rituximab was given IV on 0 d (±2 d) at a dose 375 mg/m2, to be repeated up to every three months if B cell count rises above 5% of baseline.

Rapamycin and Tacrolimus were started on −3 d (±1 d) per oral at start doses of 0.3 mg/kg QD and 0.02 mg/kg BID, respectively, and adjusted according to the plasma concentration.

Ganciclovir was given IM starting from −7 d (±2 d) at a dose of 5 mg/kg.

Prophylactic use of Chlortrimeton 0.4 mg/kg IM, and Methylprednisolone 10 mg/kg IV was administered before ATG, anti-CD40 and anti-CD20 administration to prevent infusion reactions.

Supportive administration of insulin to the STZ induced animals to support health was provided as follows:

Glargine insulin was administered QD and was administered initially at 2 U QD. The dose was increased 2 U when FBG was >150 mg/dl, and was decreased decrease 2 U when FBG was <100 mg/dl.

Insulin was administered BID, in the morning and evening according to the recorded blood glucose level of the animal. For morning doses, <200 mg/dl received no insulin, 200-350 mg/dl received 4 U insulin, 350-400 mg/dl received 6 U insulin, 400-600 mg/dl received 8 U insulin, and >600 mg/dl received 10 U insulin. For evening doses, <300 mg/dl received no insulin, 300-350 mg/dl received 4 U insulin, 350-400 mg/dl received 6 U insulin, 400-600 mg/dl received 8 U insulin, and >600 mg/dl received 10 U insulin.

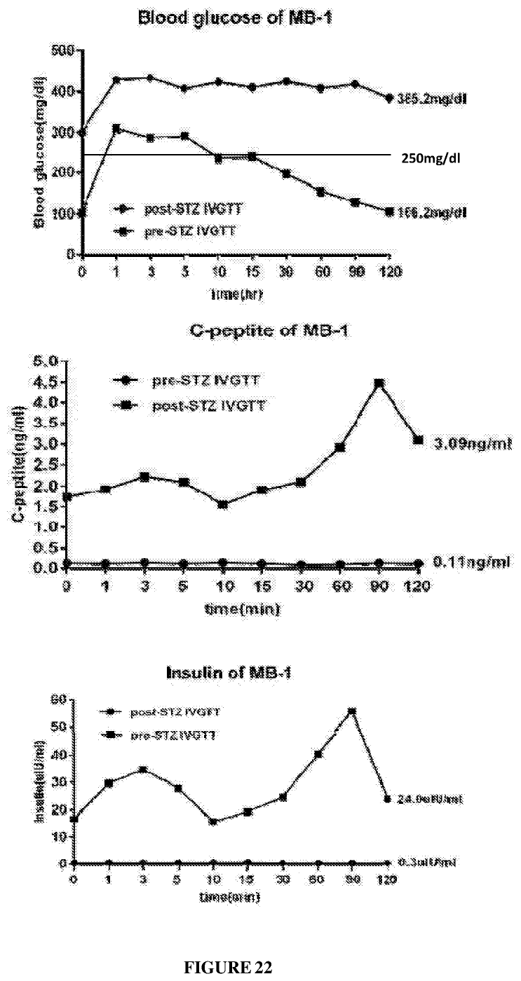

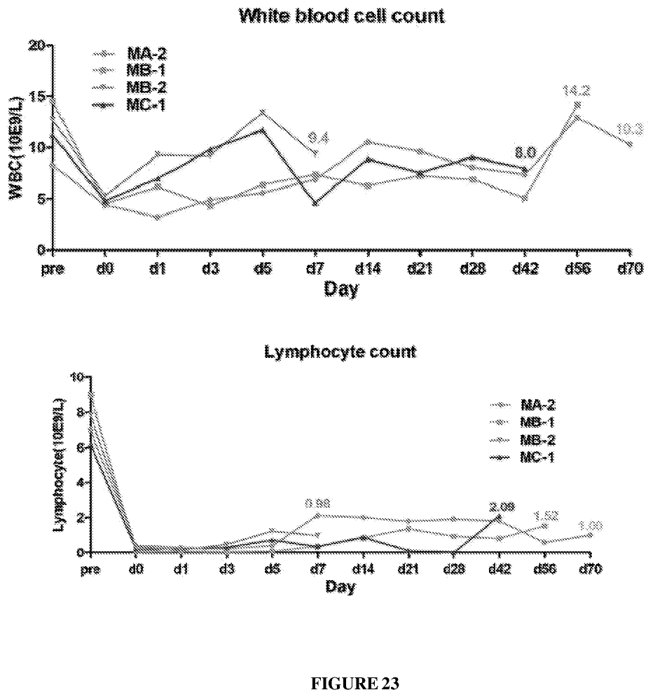

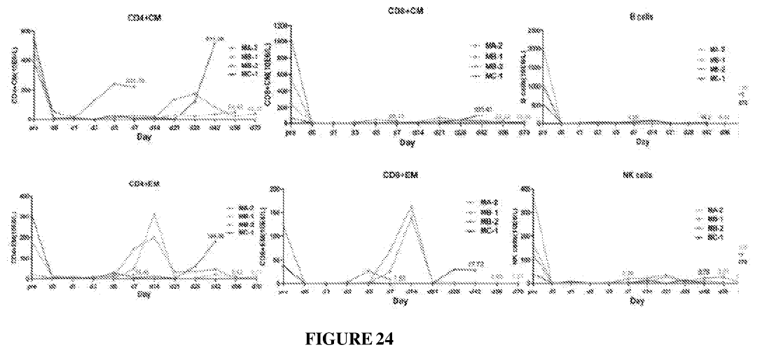

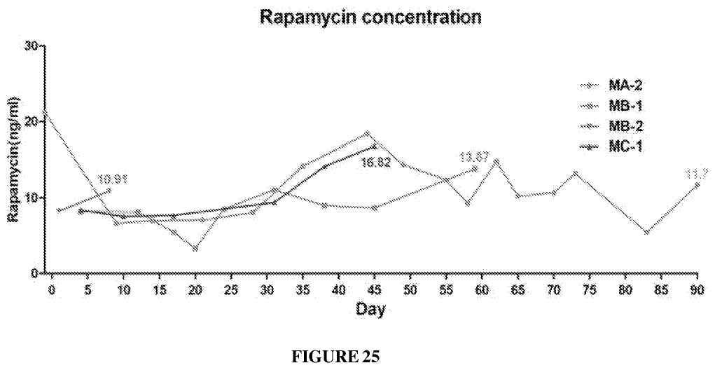

A pilot experiment using the STZ diabetes induction protocol on a monkey (MB-1) is shown in FIG. 22, where the animal is managed according to the scheme in FIG. 21. The animal was assessed for blood glucose, C-peptide, and insulin following administration of 50% dextrose 1 ml/kg iv to measure the functional output of the transplanted cells; the data in FIG. 22 indicates that the diabetes induction protocol was successful due to the increase in blood glucose and decrease in C-peptide and insulin following STZ treatment. Using this protocol, further animals MA-1, MA-2, MB-2, MC-1, MD-1, and ME-1 were induced with diabetes and transplanted with grafts according to Table 2 below. The animals were monitored for white blood cell count, lymphocyte count, CD4+ cell types, CD8+ cell types, B cells, NK cells, and Rapamycin levels following transplantation (FIGS. 23, 24, and 25).

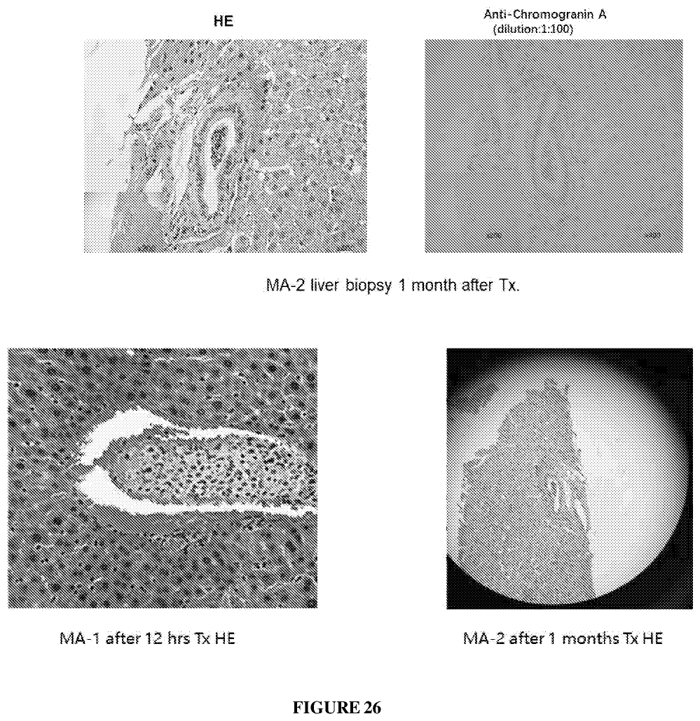

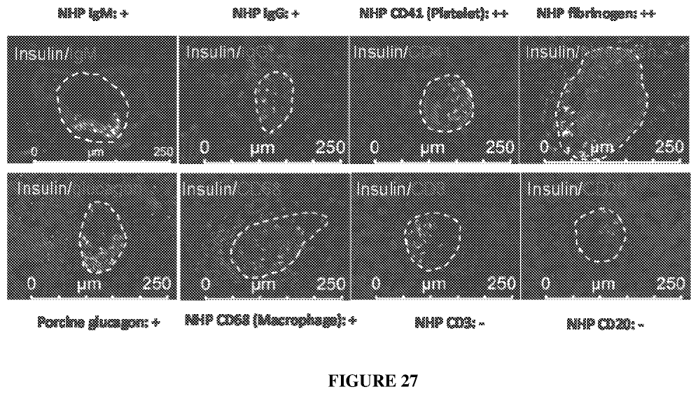

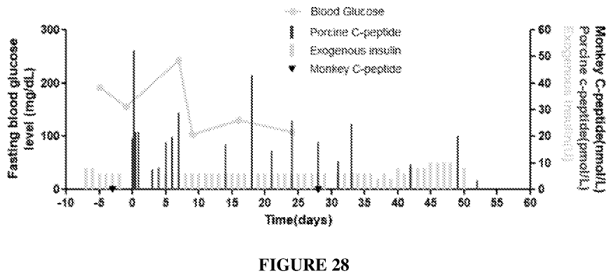

Animals MTA-1 and MA-2 were later analyzed for immunohistochemistry of liver biopsy 12 hr and 1 month after transplant for presence of the transplanted 4-7 islets. Liver biopsy was performed and stained for hematoxylin/eosin (in the case of MA-1 and MA-2) and (in the case of MA2) the neuroepithelial marker chromogranin A which stains islet cells, indicating the presence of islet-like structures and engraftment of the 4-7 cells into the animal liver (see FIG. 26).

Immunohistochemistry, blood glucose, C-peptide (both monkey and porcine), and insulin levels will continue to be monitored in all of the animals in Table 2 to assess the function of the graft in non-human primates over a longer period of time.

TABLE 2
Animals Generated for NHP Xenotransplantation Recipient Study

| | | Islets | | | | | |
|---|---|---|---|---|---|---|---|
| Recipient | | from | | | | | |
| monkey | Treatment | donor | Viability | | Purity, | Endotoxin, | |
| No. | before Tx | pigs | (beta cell %) | | IEQ/ml | EU/ml | Tx date |
| MA-1 | IS | 4-7 islets, | — | | — | <0.005 | 2018 Oct. 3 |
| | | 29k IEQ/kg | | | | | |
| MA-2 | IS | 4-7 islets, | 80% | (13%) | 1000 | <0.005 | 2018 Oct. 16 |
| | | 2k IEQ/kg | | | | | |
| MB-1 | STZ + IS | 4-7 islets, | 79.4% | (16%) | 2600 | <0.005 | 2018 Dec. 14 |
| | | 39k IEQ/kg | | | | | |
| MB-2 | STZ + IS | 4-7 islets, | 78.1% | (23.8%) | 3500 | <0.005 | 2019 Feb. 17 |
| | | 35k IEQ/kg | | | | | |
| MC-1 | STZ + IS | WT islets, | 81.6% | (24.9%) | 3900 | <0.005 | 2019 Jan. 11 |
| | | 39k IEQ/kg | | | | | |
| MB-11 | STZ + IS | WT islets, | ~80% | (~25%) | ~5000 | <0.005 | 2019 Oct. 29 |
| | | 50k IEQ/kg | | | | | |

| | | |
|---|---|---|
| MD-1 | STZ + IS | Blank |
| ME-1 | STZ | Blank |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An isolated transgenic porcine islet cell, wherein said cell:
(a) is substantially free of enzymatic activity of at least one glycosyltransferase enzyme, wherein said glycosyltransferase enzyme is GGTA, B4GALNT2, or CMAH;
(b) expresses at least two polypeptide sequences derived from a non-porcine mammalian species, wherein said at least two polypeptide sequences comprise at least two of CD46, CD55, CD59, THBD, TFPI, CD39, B2M, HLAE, CD47, A20, PD-L1, FASL, or HO-1; and
(c) exhibits one or more of the following: reduced toxicity from complement derived from said non-porcine mammalian species, reduced induction of activated protein C coagulation derived from said non-porcine mammalian species, reduced induction of thrombin-antithrombin complex derived from said non-porcine mammalian species, or reduced toxicity from NK cells derived from said non-porcine species.

2. The transgenic porcine islet cell of claim 1, wherein said cell is substantially free of enzymatic activity of at least two, or all three glycosyltransferase enzymes selected from GGTA, B4GALNT2, and CMAH.

3. The transgenic porcine islet cell of claim 1 or 2, wherein said cell expresses at least three, at least four, at least five, at least six, at least seven, at least eight, at least nine, at least ten, at least eleven, at least twelve, or all of: CD46, CD55, CD59, THBD, TFPI, CD39, B2M, HLAE, CD47, A20, PD-L1, FASL, or HO-1.

4. An isolated transgenic porcine islet cell, wherein said islet cell:
(a) is substantially free of enzymatic activity of at least two glycosyltransferase enzymes, wherein said glycosyltransferase enzymes comprise at least two of GGTA, B4GALNT2, or CMAH;
(b) expresses a polypeptide sequence derived from a non-porcine mammalian species, wherein said polypeptide sequence is CD46, CD55, CD59, THBD, TFPI, CD39, B2M, HLAE, CD47, A20, PD-L1, FASL, or HO-1; and
(c) exhibits reduced toxicity from complement derived from said non-porcine mammalian species, reduced induction of activated protein C coagulation derived from said non-porcine species, reduced induction of thrombin-antithrombin complex derived from said non-porcine species, or reduced toxicity from NK T-cells derived from said non-porcine species.

5. The transgenic porcine islet cell of claim 4, wherein said cell is substantially free of enzymatic activity of GGTA, B4GALNT2, and CMAH.

6. The transgenic porcine islet cell of claim 4 or 5, wherein said cell expresses at least two, at least three, at least four, at least five, at least six, at least seven, at least eight, at least nine, at least ten, at least eleven, at least twelve, or all of CD46, CD55, CD59, THBD, TFPI, CD39, B2M, HLAE, CD47, A20, PD-L1, FASL, or HO-1.

7. The transgenic porcine islet cell of any one of claims 1-6, expressing CD46, CD55, CD59, CD39, B2M, HLAE, and CD47.

8. The transgenic porcine islet cell of any one of claims 1-6, wherein said cell is substantially free of expression of said glycosyltransferase enzyme or enzymes.

9. The transgenic porcine islet cell of claim 8, wherein said cell comprises a frameshift mutation in said glycosyltransferase enzyme or enzymes resulting in premature termination of translation, thereby ablating activity of said glycosyltransferase enzyme.

10. The transgenic porcine islet cell of any one of claims 1-9, wherein a nucleic acid sequence or sequences encoding said polypeptide sequence or sequences derived from non-porcine mammalian species are inserted within non-orthologous loci of the porcine ortholog.

11. The transgenic porcine islet cell of any one of claims 1-10, wherein a nucleic acid sequence or sequences encoding said polypeptide sequence or sequences derived from non-porcine mammalian species are operably linked to non-orthologous promoters of the porcine ortholog.

12. The transgenic porcine islet cell of claim 11, wherein said non-orthologous promoters are non-porcine promoters.

13. The transgenic porcine islet cell of any one of claims 1-12, wherein said islet cell is derived by disaggregation of a porcine pancreas.

14. The transgenic porcine islet cell of any one of claims 1-13, wherein said islet cell is an alpha cell, a beta cell, a delta cell, an epsilon cell, a Pancreatic polypeptide (PP) cell, or any combination thereof.

15. The transgenic porcine islet cell of any one of claims 1-14, wherein said non-porcine mammalian species is a primate species.

16. The transgenic porcine islet cell of any one of claims 1-15, wherein said cell exhibits survival greater than 8 days when transplanted into said non-porcine mammalian species.

17. The transgenic porcine cell of any one of claims 1-16, wherein said cell exhibits a reduced IBMIR to PBMCs isolated from said non-porcine mammalian species.

18. A composition comprising a therapeutically effective amount of isolated porcine islet cells according to any one of claims 1-17.

19. The composition of claim 18, wherein said isotonic buffered solution further comprises heparin or a TNF-alpha inhibitor.

20. The composition of claim 18 or 19, comprising at least about 12% to about 25% beta cells or at least about 15% to about 30% alpha cells.

21. The composition of any one of claims 18-20, wherein said composition is prepared according to any one of claims 35-42.

22. A method of treating an insulin resistant or deficient condition in a non-porcine mammal in need thereof, comprising administering a therapeutically effective dose of isolated transgenic porcine islet cells according to any one of claims 1-17 or a composition according to any one of claims 18-21 to said mammal.

23. The method of claim 22, comprising centrally administering said cells via an internal jugular vein or a hepatic portal vein of said mammal.

24. The method of any one of claims 22-23, wherein said insulin resistant condition comprises type 1 diabetes mellitus.

25. The method of any one of claims 22-23, wherein said insulin resistant condition comprises type 2 diabetes mellitus.

26. The method of any one of claims 22-25, wherein said non-porcine mammal has received an induction regimen comprising therapeutically effective doses of anti-thymocyte globulin, anti-CD40 antibody, anti-CD20 antibody, a rapalog, a calcineurin inhibitor, ganciclovir or a prodrug thereof, an antihistamine, and a corticosteroid prior to administering said transgenic porcine islet cell or said composition.

27. The method of any one of claims 22-26, comprising administering therapeutically effective doses of anti-CD40 antibody, a rapalog, a calcineurin inhibitor, and ganciclovir or a prodrug thereof following administration of said transgenic porcine islet cell or said composition.

28. The method of any one of claims 22-27, comprising administering therapeutically effective doses of an intermediate- or long-acting insulin analog, insulin glargine, insulin detemir, or NPH insulin following administration of said transgenic porcine islet cell or said composition.

29. The method of any one of claims 22-28, wherein said therapeutically effective dose is at least 5,000 IEQ per kg of non-porcine mammal body weight.

30. An isolated porcine islet comprising an isolated porcine islet cell according to any one of claims 1-17.

31. An isolated porcine pancreatic organoid comprising an isolated porcine islet cell according to any one of claims 1-17.

32. The isolated porcine islet or isolated porcine pancreatic organoid of claim 30 or 31, wherein said islet or organoid is substantially free of pancreatic exocrine cells.

33. The isolated porcine pancreatic organoid of claim 31 or 32; wherein said pancreatic organoid is prepared by:
(a) isolating a pancreas from a neonatal porcine animal on neonatal day 7 or earlier; and
(b) subjecting said pancreas to mechanical or enzymatic digestion to generate organoid fragments, and optionally:
(c) purifying organoid fragments of step (b) by ficoll gradient sedimentation.

34. An isolated porcine pancreas comprising a porcine islet cell according to any one of claims 1-17.

35. A method of improving yield of islets from a porcine donor prior to transplantation to a non-porcine mammalian recipient, comprising
(a) providing pancreatic organoids from a neonatal porcine animal that have been subjected to a purification procedure;
(b) culturing said organoids in the presence of an effective concentration of a caspase inhibitor for at least 90 minutes following said purification; and
(c) continuing culture in the presence of an effective concentration of a corticosteroid for at least 7 days.

36. The method of claim 35; wherein said purification procedure comprises:
(a) isolating a pancreas from a transgenic neonatal porcine animal on neonatal day 7 or earlier; and
(b) subjecting said pancreas to mechanical or enzymatic digestion to generate organoid fragments, and optionally:
(c) purifying organoid fragments from said digested pancreas by ficoll gradient sedimentation.

37. The method of claim 35 or 36, wherein said neonatal porcine animal is a transgenic pig comprising at least one porcine cell according to any one of claims 1-17.

38. The method of any one of claims 35-37, wherein said caspase inhibitor is Z-VAD-FMK.

39. The method of any one of claims 35-38, wherein said corticosteroid is methylprednisolone.

40. The method of any one of claims 35-39, wherein said pancreatic organoids are cultured in the presence of IBMX, a phosphodiesterase inhibitor, or an adenosine receptor antagonist.

41. The method of any one of claims 35-40, wherein said pancreatic organoids are cultured in the presence of nicotinamide or a metabolically acceptable analog thereof.

42. A method of treating an insulin resistant or deficient condition in a non-porcine mammal in need thereof, comprising transplanting organoids according to any one of claims 35-41 into said non-porcine mammalian mammal when said organoids meet any of the following criteria (a) endotoxin less than about 5 EU/kg;
(b) negative gram stain;
(c) viability greater than about 70%; or
(d) islet concentration greater than or equal to about 20,000 IEQ/mL of total settled volume.
